(19) United States
(12) Patent Application Publication
(10) Pub. No.: US 2020/0348595 A1
(43) Pub. Date: Nov. 5, 2020

(54) COMPOSITION, FILM, AND PRODUCTION METHOD OF PATTERNED SUBSTRATE

(71) Applicant: JSR CORPORATION, Tokyo (JP)

(72) Inventors: Shin-ya NAKAFUJI, Tokyo (JP); Kengo EHARA, Tokyo (JP); Tomoaki TANIGUCHI, Tokyo (JP); Kazunori TAKANASHI, Tokyo (JP)

(73) Assignee: JSR CORPORATION, Tokyo (JP)

(21) Appl. No.: 16/947,119

(22) Filed: Jul. 20, 2020

Related U.S. Application Data
(63) Continuation of application No. PCT/JP2018/048360, filed on Dec. 27, 2018.

(30) Foreign Application Priority Data
Jan. 23, 2018 (JP) 2018-009030

(51) Int. Cl. G03F 7/11; C07D 209/50; C07D 471/06; C07C 233/65; C07D 251/54; C07D 405/14

(52) U.S. Cl. CPC G03F 7/11; C07D 209/50; C07D 471/06; C07C 2603/18; C07D 251/54; C07D 405/14; C07C 233/65

(57) ABSTRACT

A composition contains: a compound including at least one group selected from the group consisting of a group represented by formula (1-1), a group represented by formula (1-2), and a group represented by formula (1-3); and a solvent. In formulae (1-1) to (1-3), * and ** each denote a site bonding to a part other than the group represented by the formulae (1-1) to (1-3) in the compound; and a and b are each independently an integer of 0 to 3. In a case in which a is 0, b is no less than 1, and in a case in which a is no less than 1, b is 0.

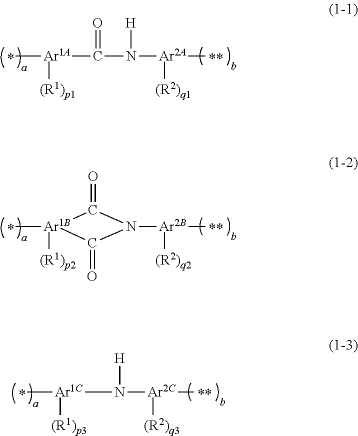

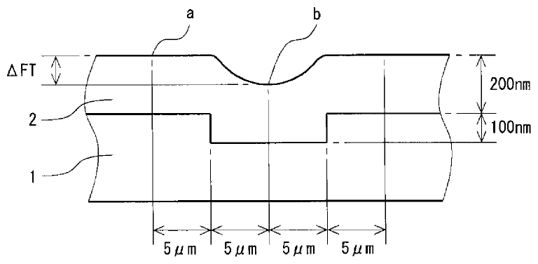

### CROSS-REFERENCE TO RELATED APPLICATIONS

The present application is a continuation application of International Application No. PCT/JP2018/048360, filed Dec. 27, 2018, which claims priority to Japanese Patent Application No. 2018-009030, filed Jan. 23, 2018. The contents of these applications are incorporated herein by reference in their entirety.

### BACKGROUND OF THE INVENTION

### Field of the Invention

The present invention relates to a composition, a film, and a production method of a patterned substrate.

### Description of the Related Art

In manufacturing semiconductor devices, resist underlayer films have been used for attaining superior integration. After a composition for resist underlayer film formation is applied directly or indirectly on at least an upper face of a substrate, a coating film thus obtained is heated to form a resist underlayer film, and then a resist pattern is formed on an upper face side of the resist underlayer film by using a resist composition or the like. Subsequently, the resist underlayer film is etched by using the resist pattern as a mask, and further, the substrate is etched by using the resultant resist underlayer film pattern as a mask, thereby enabling a desired pattern to be formed on the substrate. Accordingly, a patterned substrate can be obtained. The resist underlayer film is demanded to be superior in etching resistance.

Recently, there are increasing cases of pattern formation on a substrate having multiple types of trenches, particularly trenches with aspect ratios that are different from one another. In these cases, the composition for resist underlayer film formation is demanded to enable forming of a resist underlayer film that is superior in heat resistance and has superior flatness.

To meet these demands, a structure of and/or a functional group included in a polymer, etc. contained in the composition for resist underlayer film formation have/has been variously investigated (see Japanese Unexamined Patent Application, Publication No. 2004-177668).

### SUMMARY OF THE INVENTION

According to an aspect of the present invention, a composition includes: a compound including at least one group selected from the group consisting of a group represented by formula (1-1), a group represented by formula (1-2), and a group represented by formula (1-3), the compound having a molecular weight of no greater than 3,000; and a solvent.

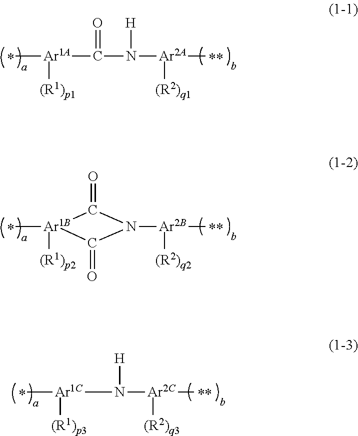

In the formulae (1-1) to (1-3), * and ** each denote a site bonding to a part other than the group represented by the formulae (1-1) to (1-3) in the compound; and a and b are each independently an integer of 0 to 3, wherein in a case in which a is 0, b is no less than 1, and in a case in which a is no less than 1, b is 0. In the formula (1-1), Ar^{1A} represents an aromatic carbocyclic group having 6 to 20 ring atoms and having a valency of (a+p1+1), or an aromatic heterocyclic group having 5 to 20 ring atoms and having a valency of (a+p1+1); R¹ represents a halogen atom, a hydroxy group, a nitro group, or a monovalent organic group having 1 to 20 carbon atoms; p1 is an integer of 0 to 11, wherein in a case in which p1 is no less than 2, a plurality of R¹s are identical or different; Ar^{2A} represents an aromatic carbocyclic group having 6 to 20 ring atoms and having a valency of (b+q1+1), or an aromatic heterocyclic group having 5 to 20 ring atoms and having a valency of (b+q1+1); R² represents a halogen atom, a hydroxy group, a nitro group, or a monovalent organic group having 1 to 20 carbon atoms; q1 is an integer of 0 to 11, wherein in a case in which q1 is no less than 2, a plurality of R²s are identical or different; a sum of p1 and a is no greater than 11; and a sum of q1 and b is no greater than 11. In the formula (1-2), Ar^{1B} represents an aromatic carbocyclic group having 6 to 20 ring atoms and having a valency of (a+p2+2), or an aromatic heterocyclic group having 5 to 20 ring atoms and having a valency of (a+p2+2); R¹ represents a halogen atom, a hydroxy group, a nitro group, or a monovalent organic group having 1 to 20 carbon atoms; p2 is an integer of 0 to 10, wherein in a case in which p2 is no less than 2, a plurality of R¹s are identical or different; Ar^{2B} represents an aromatic carbocyclic group having 6 to 20 ring atoms and having a valency of (b+q2+1), or an aromatic heterocyclic group having 5 to 20 ring atoms and having a valency of (b+q2+1); q2 is an integer of 0 to 11, wherein in a case in which q2 is 1, R² represents a halogen atom, a hydroxy group, a nitro group, or a monovalent organic group having 1 to 20 carbon atoms, and in a case in which q2 is no less than 2, a plurality of R²s are identical or different, and each R² represents a halogen atom, a hydroxy group, a nitro group, or a monovalent organic group having 1 to 20 carbon atoms, and optionally two or more of the plurality of R²s taken together represent a ring structure having 4 to 20 ring atoms together with the atom chain to which the two or more of the plurality of R²s bond; a sum of p2 and a is no greater than 10; and a sum of q2 and b is no greater than 11. In the formula (1-3), Ar^{1C} represents an aromatic carbocyclic group having 6 to 20 ring atoms and having a valency of (a+p3+1), or an aromatic heterocyclic group having 5 to 20 ring atoms and having a valency of (a+p3+1); R¹ represents a halogen atom, a hydroxy group, a nitro group, or a monovalent organic group having 1 to 20 carbon atoms; p3 is an integer of 0 to 11, wherein in a case in which p3 is no less than 2, a plurality of R¹s are identical or different; Ar^{2C} represents an aromatic heterocyclic group having 5 to 20 ring atoms and having a valency of (b+q3+1); R² represents a halogen atom, a hydroxy group, a nitro group, or a monovalent organic group having 1 to 20 carbon atoms; q3 is an integer of 0 to 11, wherein in a case in which q3 is no less than 2, a plurality of R²s are identical or different; a sum of p3 and a is no greater than 11; and a sum of q3 and b is no greater than 11.

According to another aspect of the present invention, a film is formed from one of the above-mentioned compositions.

According to a further aspect of the present invention, a production method of a patterned substrate includes applying one of the above-mentioned compositions directly or indirectly on at least an upper face of a substrate, to form a resist underlayer film. A resist pattern is formed on an upper face side of the resist underlayer film. Etching is carried out using the resist pattern as a mask.

### BRIEF DESCRIPTION OF THE DRAWING

The Figure is a schematic cross sectional view illustrating a method for evaluating flatness.

### DESCRIPTION OF EMBODIMENTS

According to one embodiment of the invention, a composition for resist underlayer film formation contains: a compound (hereinafter, may be also referred to as “(A) compound” or “compound (A)”) having a group represented by any one of the following formulae (1-1) to (1-3); and a solvent (hereinafter, may be also referred to as “(B) solvent” or “solvent (B)”).

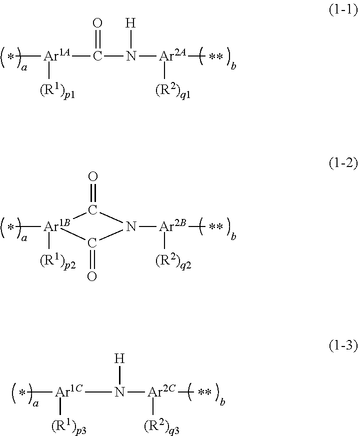

wherein,

in the formulae (1-1) to (1-3), * and ** each denote a site bonding to a part other than the group represented by the formulae (1-1) to (1-3) in the compound; and “a” and b are each independently an integer of 0 to 3, wherein in a case in which “a” is 0, b is no less than 1, and in a case in which “a” is no less than 1, b is 0,

in the formula (1-1), Ar^{1A} represents an aromatic carbocyclic group having 6 to 20 ring atoms and having a valency of (a+p1+1), or an aromatic heterocyclic group having 5 to 20 ring atoms and having a valency of (a+p1+1); R¹ represents a halogen atom, a hydroxy group, a nitro group, or a monovalent organic group having 1 to 20 carbon atoms; p1 is an integer of 0 to 11, wherein in a case in which p1 is no less than 2, a plurality of R¹s are identical or different; Ar^{2A} represents an aromatic carbocyclic group having 6 to 20 ring atoms and having a valency of (b+q1+1), or an aromatic heterocyclic group having 5 to 20 ring atoms and having a valency of (b+q1+1); R² represents a halogen atom, a hydroxy group, a nitro group, or a monovalent organic group having 1 to 20 carbon atoms; q1 is an integer of 0 to 11, wherein in a case in which q1 is no less than 2, a plurality of R²s are identical or different; a sum of p1 and “a” is no greater than 11; and a sum of q1 and b is no greater than 11,

in the formula (1-2), Ar^{1B} represents an aromatic carbocyclic group having 6 to 20 ring atoms and having a valency of (a+p2+2), or an aromatic heterocyclic group having 5 to 20 ring atoms and having a valency of (a+p2+2); R¹ represents a halogen atom, a hydroxy group, a nitro group, or a monovalent organic group having 1 to 20 carbon atoms; p2 is an integer of 0 to 10, wherein in a case in which p2 is no less than 2, a plurality of R¹s are identical or different; Ar^{2B} represents an aromatic carbocyclic group having 6 to 20 ring atoms and having a valency of (b+q2+1), or an aromatic heterocyclic group having 5 to 20 ring atoms and having a valency of (b+q2+1); q2 is an integer of 0 to 11, wherein in a case in which q2 is 1, R² represents a halogen atom, a hydroxy group, a nitro group, or a monovalent organic group having 1 to 20 carbon atoms, or in a case in which q2 is no less than 2, a plurality of R²s are identical or different, and each R² represents a halogen atom, a hydroxy group, a nitro group, or a monovalent organic group having 1 to 20 carbon atoms, or two or more of the plurality of R²s taken together represent a ring structure having 4 to 20 ring atoms together with the atom chain to which the two or more of the plurality of R²s bond; a sum of p2 and “a” is no greater than 10; and a sum of q2 and b is no greater than 11, and

in the formula (1-3), Ar^{1C} represents an aromatic carbocyclic group having 6 to 20 ring atoms and having a valency of (a+p3+1), or an aromatic heterocyclic group having 5 to 20 ring atoms and having a valency of (a+p3+1); R¹ represents a halogen atom, a hydroxy group, a nitro group, or a monovalent organic group having 1 to 20 carbon atoms; p3 is an integer of 0 to 11, wherein in a case in which p3 is no less than 2, a plurality of R¹s are identical or different; Ar^{2C} represents an aromatic heterocyclic group having 5 to 20 ring atoms and having a valency of (b+q3+1); R² represents a halogen atom, a hydroxy group, a nitro group, or a monovalent organic group having 1 to 20 carbon atoms; q3 is an integer of 0 to 11, wherein in a case in which q3 is no less than 2, a plurality of R²s are identical or different; a sum of p3 and “a” is no greater than 11; and a sum of q3 and b is no greater than 11.

According to another embodiment of the present invention, a resist underlayer film is formed from the composition for resist underlayer film formation of the one embodiment of the invention.

According to still another embodiment of the present invention, a resist underlayer film-forming method includes a step of applying a composition for resist underlayer film formation containing the compound (A) and the solvent (B) directly or indirectly on at least an upper face of a substrate.

According to yet another embodiment of the present invention, a patterned substrate-producing method includes:

a step of applying a composition for resist underlayer film formation containing the compound (A) and the solvent (B), directly or indirectly on at least an upper face of a substrate;

a step of forming a resist pattern on an upper face side of a resist underlayer film formed by the applying; and

carrying out etching using the resist pattern as a mask.

A procedure of forming on the resist underlayer film a silicon-containing film as an intermediate layer in a multilayer resist process has been investigated more recently; however, generation of defects such as cracks and peeling on the surface of the silicon-containing film leads to a requirement for superior inhibitory property on film defects.

The composition for resist underlayer film formation of the one embodiment of the present invention enables forming of a resist underlayer film that is superior in etching resistance, heat resistance, flatness and an inhibitory property on film defects. The resist underlayer film of the another embodiment of the present invention is superior in etching resistance, heat resistance, flatness and an inhibitory property on film defects. A method of forming resist film of the still another embodiment of the present invention enables a resist underlayer film that is superior in etching resistance, heat resistance, flatness and an inhibitory property on film defects to be easily and reliably formed. According to the production method of a patterned substrate of the yet another embodiment of the present invention, by using such a superior resist underlayer film, obtaining a favorable patterned substrate is enabled. Therefore, these can be suitably used for manufacture of semiconductor devices and the like, for which microfabrication is expected to progress further hereafter.

### Composition for Resist Underlayer Film Formation

The composition for resist underlayer film formation of one embodiment of the present invention (hereinafter, may be also referred to merely as “composition”) contains the compound (A) and the solvent (B). The composition may also contain optional component(s) within a range not leading to impairment of the effects of the present invention. Each component will be described below.

### (A) Compound

The compound (A) has a group represented by any one of the following formulae (1-1) to (1-3) (hereinafter, a group represented by the formula (1-1) may be also referred to as “group (I-1)”, a group represented by the formula (1-2) may be also referred to as “group (I-2)” and a group represented by the formula (1-3) may be also referred to as “group (I-3)”, and the groups (I-1) to (I-3) may be also referred to as “group (I)” as a whole). The compound (A) may have one, or a plurality of the group (I).

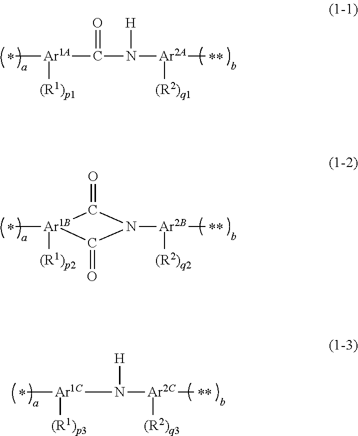

In the above formulae (1-1) to (1-3), * and ** each denote a site bonding to a part other than the group represented by the formulae (1-1) to (1-3) in the compound; and “a” and b are each independently an integer of 0 to 3, wherein in a case in which “a” is 0, b is no less than 1, and in a case in which “a” is no less than 1, b is 0.

In the above formula (1-1), Ar^{1A} represents an aromatic carbocyclic group having 6 to 20 ring atoms and having a valency of (a+p1+1), or an aromatic heterocyclic group having 5 to 20 ring atoms and having a valency of (a+p1+1); R¹ represents a halogen atom, a hydroxy group, a nitro group, or a monovalent organic group having 1 to 20 carbon atoms; p1 is an integer of 0 to 11, wherein in a case in which p1 is no less than 2, a plurality of R¹s are identical or different; Ar^{2A} represents an aromatic carbocyclic group having 6 to 20 ring atoms and having a valency of (b+q1+1), or an aromatic heterocyclic group having 5 to 20 ring atoms and having a valency of (b+q1+1); R² represents a halogen atom, a hydroxy group, a nitro group, or a monovalent organic group having 1 to 20 carbon atoms; q1 is an integer of 0 to 11, wherein in a case in which q1 is no less than 2, a plurality of R²s are identical or different; a sum of p1 and “a” is no greater than 11; and a sum of q1 and b is no greater than 11, in a case in which p1 is no less than 2, two or more of a plurality of R¹s taken together may represent a ring structure having 4 to 20 ring atoms together with the atom chain to which the two or more of the plurality of R¹s bond, and in a case in which q1 is no less than 2, two or more of the plurality of R²s taken together may represent a ring structure having 4 to 20 ring atoms together with the atom chain to which the two or more of the plurality of R²s bond.

In the above formula (1-2), Ar^{1B} represents an aromatic carbocyclic group having 6 to 20 ring atoms and having a valency of (a+p2+2), or an aromatic heterocyclic group having 5 to 20 ring atoms and having a valency of (a+p2+2); R¹ represents a halogen atom, a hydroxy group, a nitro group, or a monovalent organic group having 1 to 20 carbon atoms; p2 is an integer of 0 to 10, wherein in a case in which p2 is no less than 2, a plurality of R¹s are identical or different; Ar^{2B} represents an aromatic carbocyclic group having 6 to 20 ring atoms and having a valency of (b+q2+1), or an aromatic heterocyclic group having 5 to 20 ring atoms and having a valency of (b+q2+1); q2 is an integer of 0 to 11, wherein in a case in which q2 is 1, R² represents a halogen atom, a hydroxy group, a nitro group, or a monovalent organic group having 1 to 20 carbon atoms, or in a case in which q2 is no less than 2, a plurality of R²s are identical or different, and each R² represents a halogen atom, a hydroxy group, a nitro group, or a monovalent organic group having 1 to 20 carbon atoms, or two or more of the plurality of R²s taken together represent a ring structure having 4 to 20 ring atoms together with the atom chain to which the two or more of the plurality of R²s bond; a sum of p2 and “a” is no greater than 10; and a sum of q2 and b is no greater than 11, wherein in a case in which p2 is no less than 2, two or more of a plurality of R¹s taken together may represent a ring structure having 4 to 20 ring atoms together with the atom chain to which the two or more of the plurality of R¹s bond.

In the above formula (1-3), Ar^{1C} represents an aromatic carbocyclic group having 6 to 20 ring atoms and having a valency of (a+p3+1), or an aromatic heterocyclic group having 5 to 20 ring atoms and having a valency of (a+p3+1); R¹ represents a halogen atom, a hydroxy group, a nitro group, or a monovalent organic group having 1 to 20 carbon atoms; p3 is an integer of 0 to 11, wherein in a case in which p3 is no less than 2, a plurality of R¹s are identical or different; Ar^{2C} represents an aromatic heterocyclic group having 5 to 20 ring atoms and having a valency of (b+q3+1); R² represents a halogen atom, a hydroxy group, a nitro group, or a monovalent organic group having 1 to 20 carbon atoms; q3 is an integer of 0 to 11, wherein in a case in which q3 is no less than 2, a plurality of R²s are identical or different; a sum of p3 and “a” is no greater than 11; and a sum of q3 and b is no greater than 11, wherein in a case in which p3 is no less than 2, two or more of a plurality of R¹s taken together may represent a ring structure having 4 to 20 ring atoms together with the atom chain to which the two or more of the plurality of R¹s bond, and in a case in which q3 is no less than 2, two or more of a plurality of R²s taken together may represent a ring structure having 4 to 20 ring atoms together with the atom chain to which the two or more of the plurality of R²s bond.

Due to containing the compound (A), the composition enables forming of a resist underlayer film that is superior in etching resistance, heat resistance, flatness and an inhibitory property on film defects. Although not necessarily clarified and without wishing to be bound by any theory, the reason for achieving the effects described above due to the composition having the constitution described above may be supposed as in the following, for example. To explain specifically, the compound (A) has a specific structure that includes: an amide group to which two aromatic rings (aromatic carbon rings or aromatic heterorings) bond; an imide group to which two aromatic rings bond; and/or an amino group to which two aromatic rings bond, where at least one of the two aromatic rings is an aromatic heteroring. In the compound (A), resulting from such a specific structure, strong bonding between constitutive atoms is provided, thereby leading to potent interactions between/among molecules. When such a compound (A) is used, etching resistance of the resist underlayer film is improved, accompanied by a decrease in sublimability and the like of the compound (A), whereby heat resistance and the inhibitory property on film defects of the resist underlayer film are improved. In addition, it is considered that fluidity of the compound (A) at a high temperature can be reduced due to the specific structure described above, and as a result, flatness of the resist underlayer film is improved.

The “aromatic carbocyclic group” as referred to herein means a group obtained by removing from an arene, hydrogen atom(s) on one or a plurality of aromatic rings. Examples of the arene that gives the aromatic carbocyclic group having 6 to 20 ring atoms which may be represented by Ar^{1A}, Ar^{2A}, Ar^{1B}, Ar^{2B}, or Ar^{1C} include benzene, toluene, xylene, naphthalene, anthracene, phenanthrene, tetracene, pyrene, triphenylene, perylene and the like. Of these, benzene or naphthalene is preferred.

The “aromatic heterocyclic group” as referred to herein means a group obtained by removing from a heteroarene, hydrogen atom(s) on one or a plurality of aromatic rings. Examples of the heteroarene that gives the aromatic heterocyclic group having 5 to 20 ring atoms which may be represented by Ar^{1A}, Ar^{2A}, Ar^{1B}, Ar^{2B}, Ar^{1C} or Ar^{2C} include: nitrogen atom-containing heterocyclic compounds such as pyridine, quinoline, isoquinoline, indole, pyrazine, pyrimidine, pyridazine, and triazine; oxygen atom-containing heterocyclic compounds such as furan, pyran, benzofuran, and benzopyran; sulfur atom-containing heterocyclic compounds such as thiophene and benzothiophene; and the like. Of these, the nitrogen atom-containing heterocyclic compound is preferred, and triazine is more preferred.

It is preferred that at least one of Ar^{1A} and Ar^{2B} in the above formula (1-1), and at least one of Ar^{1B} and Ar^{2B} in the above formula (1-2) are each the aromatic carbocyclic group, and it is more preferred that both Ar^{1A} and Ar^{2B}, and both Ar^{1B} and Ar^{2B} each represent the aromatic carbocyclic group. Ar^{1C} in the above formula (1-3) represents preferably the aromatic carbocyclic group. When the group described above in the compound (A) is the aromatic carbocyclic group in this manner, etching resistance, heat resistance, flatness, and the inhibitory property on film defects of the resist underlayer film can be further improved.

The monovalent organic group having 1 to 20 carbon atoms which may be represented by R¹ or R² in the above formulae (1-1) to (1-3) is exemplified by: a monovalent hydrocarbon group having 1 to 20 carbon atoms; a group having 1 to 20 carbon atoms and having a hetero atom obtained by including a divalent hetero atom-containing group between two adjacent carbon atoms of the monovalent hydrocarbon group having 1 to 20 carbon atoms; a group obtained by substituting with a monovalent hetero atom-containing group, a part or all of hydrogen atoms of the monovalent hydrocarbon group having 1 to 20 carbon atoms or of the group having 1 to 20 carbon atoms and having a hetero atom; and the like.

The “hydrocarbon group” as referred to herein is exemplified by a chain hydrocarbon group, an alicyclic hydrocarbon group and an aromatic hydrocarbon group. The “hydrocarbon group” may be either a saturated hydrocarbon group or an unsaturated hydrocarbon group. The “chain hydrocarbon group” as referred to herein means a hydrocarbon group not including a cyclic structure but being constituted with only a chain structure, and both a linear hydrocarbon group and a branched hydrocarbon group may be included. The “alicyclic hydrocarbon group” as referred to herein means a hydrocarbon group that includes, as a ring structure, not an aromatic ring structure but an alicyclic structure alone, and may include both a monocyclic alicyclic hydrocarbon group and a polycyclic alicyclic hydrocarbon group. However, it is not necessary for the alicyclic hydrocarbon group to be constituted with only an alicyclic structure; it may include a chain structure in a part thereof. The “aromatic hydrocarbon group” as referred to herein means a hydrocarbon group that includes an aromatic ring structure as a ring structure. However, it is not necessary for the aromatic hydrocarbon group to be constituted with only an aromatic ring structure; it may include a chain structure or an alicyclic structure in a part thereof.

The monovalent hydrocarbon group having 1 to 20 carbon atoms is exemplified by a monovalent chain hydrocarbon group having 1 to 20 carbon atoms, a monovalent alicyclic hydrocarbon group having 3 to 20 carbon atoms, a monovalent aromatic hydrocarbon group having 6 to 20 carbon atoms, and the like.

Examples of the monovalent chain hydrocarbon group having 1 to 20 carbon atoms include:

chain saturated hydrocarbon groups, e.g., alkyl groups such as a methyl group, an ethyl group, a n-propyl group, an i-propyl group, a sec-butyl group, and a t-butyl group;

chain unsaturated hydrocarbon groups, e.g.,

alkenyl groups such as an ethenyl group, a 1-propenyl group, an allyl group, and a butenyl group, and

alkynyl groups such as an ethynyl group, a propynyl group, and a butynyl group; and the like.

Examples of the monovalent alicyclic hydrocarbon group having 3 to 20 carbon atoms include:

alicyclic saturated hydrocarbon groups, e.g.,

monocyclic alicyclic saturated hydrocarbon groups such as a cyclopentyl group and a cyclohexyl group, and

polycyclic alicyclic saturated hydrocarbon groups such as a norbornyl group, an adamantyl group, and a tricyclodecyl group;

alicyclic unsaturated hydrocarbon groups, e.g.,

monocyclic alicyclic unsaturated hydrocarbon groups such as a cyclopentenyl group and a cyclohexenyl group, and

polycyclic alicyclic unsaturated hydrocarbon groups such as a norbornenyl group and a tricyclodecenyl group; and the like.

Examples of the monovalent aromatic hydrocarbon group having 6 to 20 carbon atoms include:

aryl groups such as a phenyl group, a tolyl group, a xylyl group, a naphthyl group, and an anthryl group;

aralkyl groups such as a benzyl group, a phenethyl group, a naphthylmethyl group, and an anthryl methyl group; and the like.

Examples of the hetero atom constituting the monovalent or divalent hetero atom-containing group include an oxygen atom, a nitrogen atom, a sulfur atom, a phosphorus atom, a silicon atom, a halogen atom, and the like. Examples of the halogen atom include a fluorine atom, a chlorine atom, a bromine atom, an iodine atom, and the like.

The divalent hetero atom-containing group is exemplified by —O—, —CO—, —S—, —CS—, —NR′—, a group obtained by combining two or more of these, and the like, wherein R′ represents a hydrogen atom or a monovalent hydrocarbon group.

Examples of the monovalent hetero atom-containing group include halogen atoms such as a fluorine atom, a chlorine atom, a bromine atom and an iodine atom, a hydroxy group, a carboxy group, a cyano group, an amino group, a sulfanyl group (—SH), and the like.

Examples of the ring structure having 4 to 20 ring atoms which may be constituted from two or more of the plurality of R¹ or two or more of the plurality of R²s include: alicyclic structures such as a cyclobutane structure, a cyclopentane structure, a cyclohexane structure, a cyclobutene structure, a cyclopentene structure, and a cyclohexene structure, and the like.

In the above formulae (1-1) to (1-3), one of “a” and b is 0. In other words, the group (I) is positioned at one end of the molecule of the compound (A). In the case in which “a” is 0, b is preferably 1 or 2. In the case in which b is 0, “a” is preferably 1 or 2.

It is preferred that: at least one of p1 and q1 in the above formula (1-1), at least one of p2 and q2 in the above formula (1-2), and at least one of p3 and q3 in the above formula (1-3) are each no less than 1, and that at least one of R¹ and R² represents the organic group described above. Due to thus having the at least one organic group on the aromatic ring thereof, the compound (A) enables etching resistance, heat resistance, flatness, and the inhibitory property on film defects of the resist underlayer film to be further improved.

In the case in which b is 0, p1, p2 and p3 are each preferably 0 to 2, more preferably 0 or 1, and still more preferably 0. Meanwhile, q1, q2 and q3 are each preferably 1 to 3, more preferably 1 or 2, and still more preferably 1.

In the case in which “a” is 0, p1, p2 and p3 are each preferably 1 to 3, more preferably 1 or 2, and still more preferably 1. Meanwhile, q1, q2 and q3 are each preferably 0 to 2, more preferably 0 or 1, and still more preferably 0.

In the case in which at least one of R¹ and R² represents the organic group, at least one of the organic group is preferably a multiple bond-containing group. When the multiple bond-containing group is included as R¹ or R², a percentage content of hydrogen atoms in the compound (A) can be further decreased, and as a result, etching resistance of the resist underlayer film can be further improved. The term “multiple bond-containing group” as referred to herein means a group that includes a double bond or a triple bond between two atoms. The double bond may include a conjugated double bond in an aromatic carbon ring or an aromatic heteroring.

The multiple bond-containing group is exemplified by a carbon-carbon double bond-containing group, a carbon-carbon triple bond-containing group, a carbon-nitrogen double bond-containing group, a carbon-nitrogen triple bond-containing group, a carbon-oxygen double bond-containing group, and the like.

Examples of the carbon-carbon double bond-containing group include: ethylenic double bond-containing groups such as a vinyl group, a vinyloxy group, an allyl group, an allyloxy group, a (meth)acryloyl group, and a (meth)acryloyloxy group; aromatic hydrocarbon groups such as a phenyl group and a naphthyl group; groups obtained from these groups by substituting a part or all of hydrogen atoms with a hydroxy group, a halogen atom, a monovalent organic group or the like (hereinafter, these may be also referred to as “substituent (a)”); and the like.

Examples of the carbon-carbon triple bond-containing group include: a propargyl group, a propargyloxy group; groups obtained from these groups by substituting a part or all of hydrogen atoms with the substituent (a); an ethynyl group; an ethynyloxy group; an ethynylcarbonyl group; a phenylethynylcarbonyl group; and the like.

Examples of the carbon-nitrogen double bond-containing group include: imino-containing groups such as a methylimino group; nitrogen-containing heterocyclic groups such as a pyridyl group, a pyrazinyl group, a pyrimidinyl group, and a triazinyl group; groups obtained from these groups by substituting a part or all of hydrogen atoms with the substituent (a); and the like.

Examples of the carbon-nitrogen triple bond-containing group include: cyanoalkyl groups such as a cyanomethyl group; cyanoalkyloxy groups such as a cyanomethyloxy group; cyanoaryl groups such as a cyanophenyl group; cyanoaryloxy groups such as a cyanophenyloxy group; groups obtained from these groups by substituting a part or all of hydrogen atoms with the substituent (a); a cyano group; a cyanate group; and the like.

Examples of the carbon-oxygen double bond-containing group include: acyl groups such as a formyl group and an acetyl group; acyloxy groups such as a formyloxy group and an acetyloxy group; alkoxycarbonyl groups such as a methoxycarbonyl group: aryloxycarbonyl groups such as a phenoxycarbonyl group; groups obtained from these groups by substituting a part or all of hydrogen atoms with the substituent (a); and the like.

The multiple bond-containing group is preferably the carbon-carbon triple bond-containing group, and more preferably an ethynyl group or a phenylethynylcarbonyl group.

It is also preferred that the organic group which may be represented by R¹ or R² is a crosslinkable functional group. When the compound (A) has the crosslinkable functional group, crosslinking reactivity may be further improved, and as a result, etching resistance and heat resistance of the resist underlayer film can be further improved. The “crosslinkable functional group” as referred to herein means a functional group that is capable of forming a crosslinking bond that crosslinks in the compound (A), between the compounds (A) or between the compound (A) and an other compound by a reaction between the crosslinkable functional groups or a reaction of the crosslinkable functional group with an other functional group.

The crosslinkable functional group is exemplified by the ethylenic double bond-containing group, the carbon-carbon triple bond-containing group, the imino-containing group, the carbon-nitrogen triple bond-containing group, the acyl group, and the acyloxy group, as well as, for example, an epoxy group, a dioxole group, a hydroxy chain hydrocarbon group, an aromatic hydroxy group-containing group, an amino group, a substituted amino group, and the like.

Examples of the epoxy group include: oxirane ring-containing groups such as an oxiranyl group, an oxiranylmethyl group, and an oxiranylmethyloxy group; oxetane ring-containing groups such as an oxetanyl group, an oxetanylmethyl group, and an oxetanylmethyloxy group; groups obtained from these groups by substituting a part or all of hydrogen atoms with the substituent (a); and the like.

Examples of the dioxole group include —O—CR^{a}R^{b}—O—, —O—CR^{a}R^{b}—O—CR^{a}R^{b}—, —O—CR^{a}R^{b}—CR^{a}R^{b}—O—, and the like, wherein R^{a} and R^{b} each independently represent a hydrogen atom, a fluorine atom, a monovalent hydrocarbon group having 1 to 20 carbon atoms, or a monovalent fluorinated hydrocarbon group having 1 to 20 carbon atoms, or R^{a} and R^{b} taken together represent an alicyclic structure having 3 to 20 ring atoms together with the carbon atom to which R^{a} and R^{b} bond.

Examples of the hydroxy chain hydrocarbon group include hydroxyalkyl groups such as a hydroxymethyl group, a 1-hydroxyethyl group, a 1-hydroxypropyl group, and a 2-hydroxy-2-propyl group, and the like.

Examples of the aromatic hydroxy group-containing group include a hydroxybutadienediyl group, a hydroxyphenyl group, a hydroxynaphthyl group, a hydroxyanthryl group, and the like.

Examples of the substituted amino group include: monoalkylamino groups such as a methylamino group and an ethylamino group; dialkylamino groups such as a dimethylamino group and a diethylamino group; and the like.

The crosslinkable functional group is preferably the dioxole group, the hydroxy chain hydrocarbon group, the aromatic hydroxy group-containing group, or the substituted amino group, and more preferably —O—CH₂—O—, a hydroxymethyl group, a hydroxybutadienediyl group or a dimethylamino group.

The group (I-1) is exemplified by groups represented by the following formulae (1-1-1) to (1-1-4) and the like; the group (I-2) is exemplified by groups represented by the following formulae (1-2-1) to (1-2-3) and the like; and the group (I-3) is exemplified by a group represented by the following formula (1-3-1) and the like.

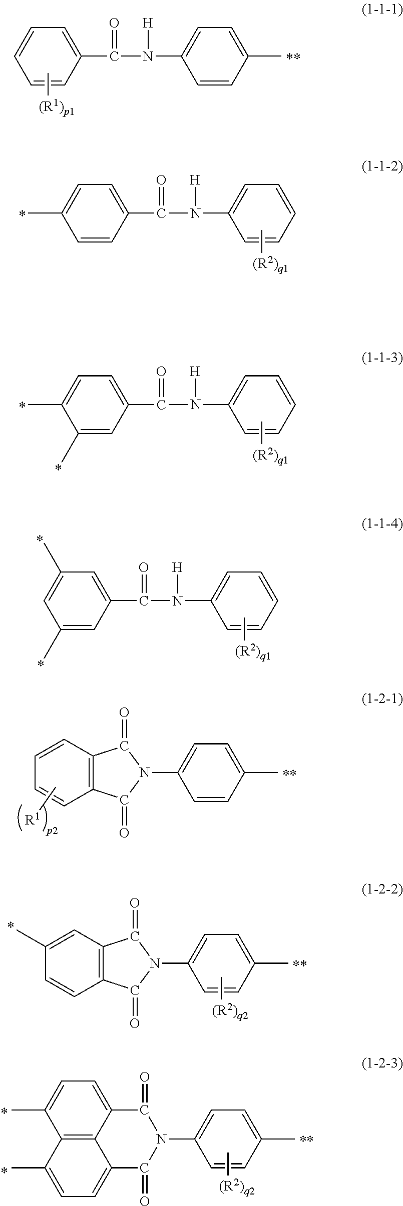

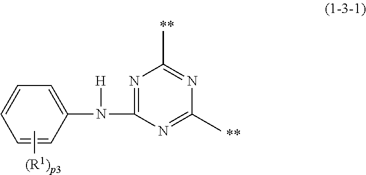

In the above formulae (1-1-1) to (1-3-1), R¹, R², p¹, q¹, p², q², p³, *, and ** are as defined in the above formulae (1-1) to (1-3).

The lower limit of a number of the group (I) included in the compound (A) is preferably 2. The upper limit of the number of the group (I) is preferably 10, and more preferably 5.

The compound (A) is exemplified by compounds represented by the following formula (2-1) or (2-2), and the like.

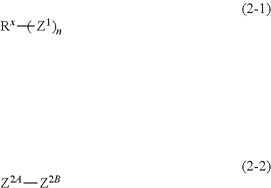

In the above formula (2-1), Z¹ represents the group (I) having a valency of c, wherein c is an integer of 1 to 3; n is an integer of 1 to 10, and in a case in which n is no less than 2, a plurality of Z¹s may be identical or different; and R^{X} represents an organic group having 1 to 40 carbon atoms and having a valency of m, wherein m is a sum of c's for n Z¹s.

In the above formula (2-2), Z^{2A} and Z^{2B} each independently represent the group (I) having a valency of d; and d is an integer of 1 to 3.

Z¹ is an example of the group (I), wherein “a” orb in the above formulae (1-1) to (1-3) is c. The organic group, which is represented by R^{X}, having 1 to 40 carbon atoms and having a valency of m is exemplified by a group obtained by removing (m-1) hydrogen atom(s) from the monovalent organic group which may be represented by R¹ or R², and the like.

Z^{2A} and Z^{2B} are examples of the group (I), wherein “a” orb in the above formulae (1-1) to (1-3) is d.

R^{X} in the above formula (2-1) is exemplified by: groups represented by the following formulae (3-1-1) to (3-1-4) and the like, wherein m is 2; a group represented by the following formula (3-2-1) and the like, wherein m is 3; and groups represented by the following formulae (3-3-1) and (3-3-2) and the like, wherein m is 4.

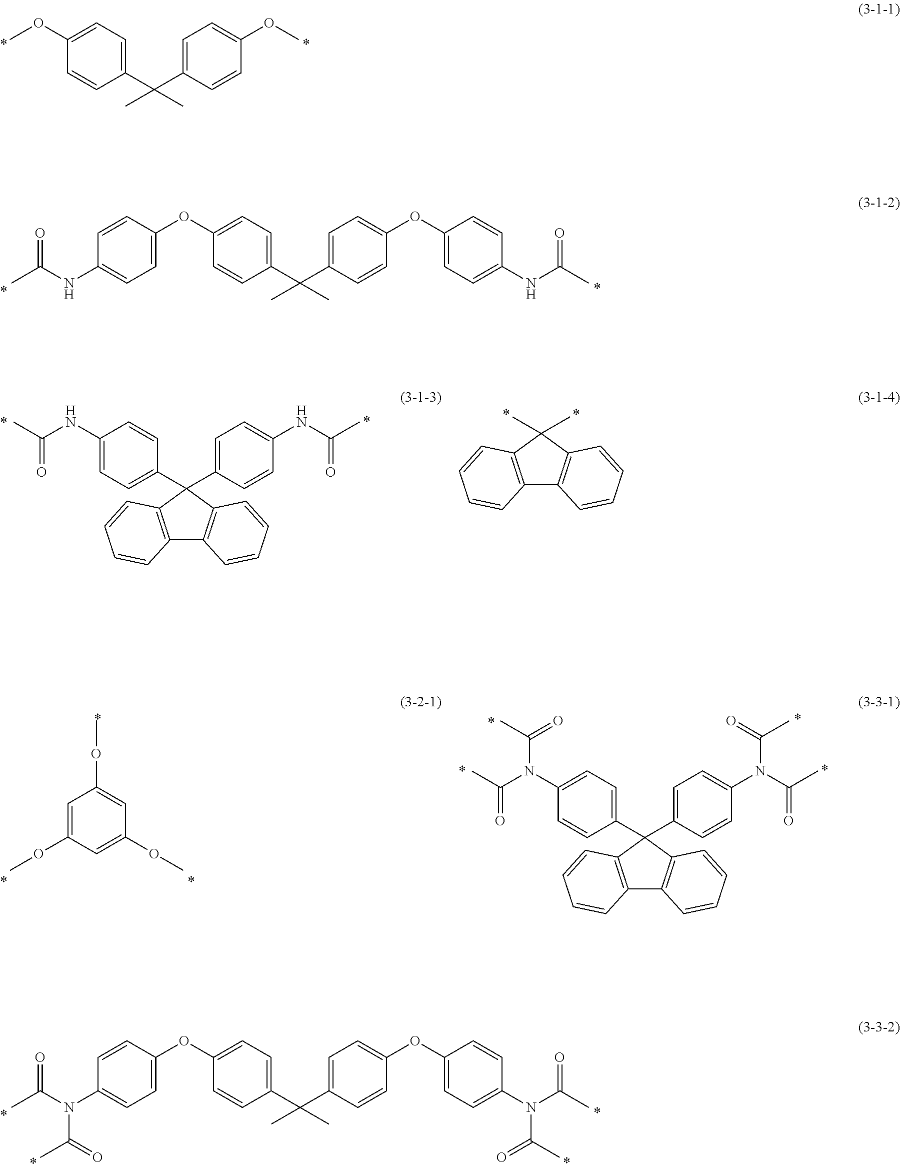

In the above formulae (3-1-1) to (3-3-2), * denotes a site bonding to Z¹.

The compound (A) is exemplified by compounds represented by the following formulae (i-1) to (i-11), and the like.

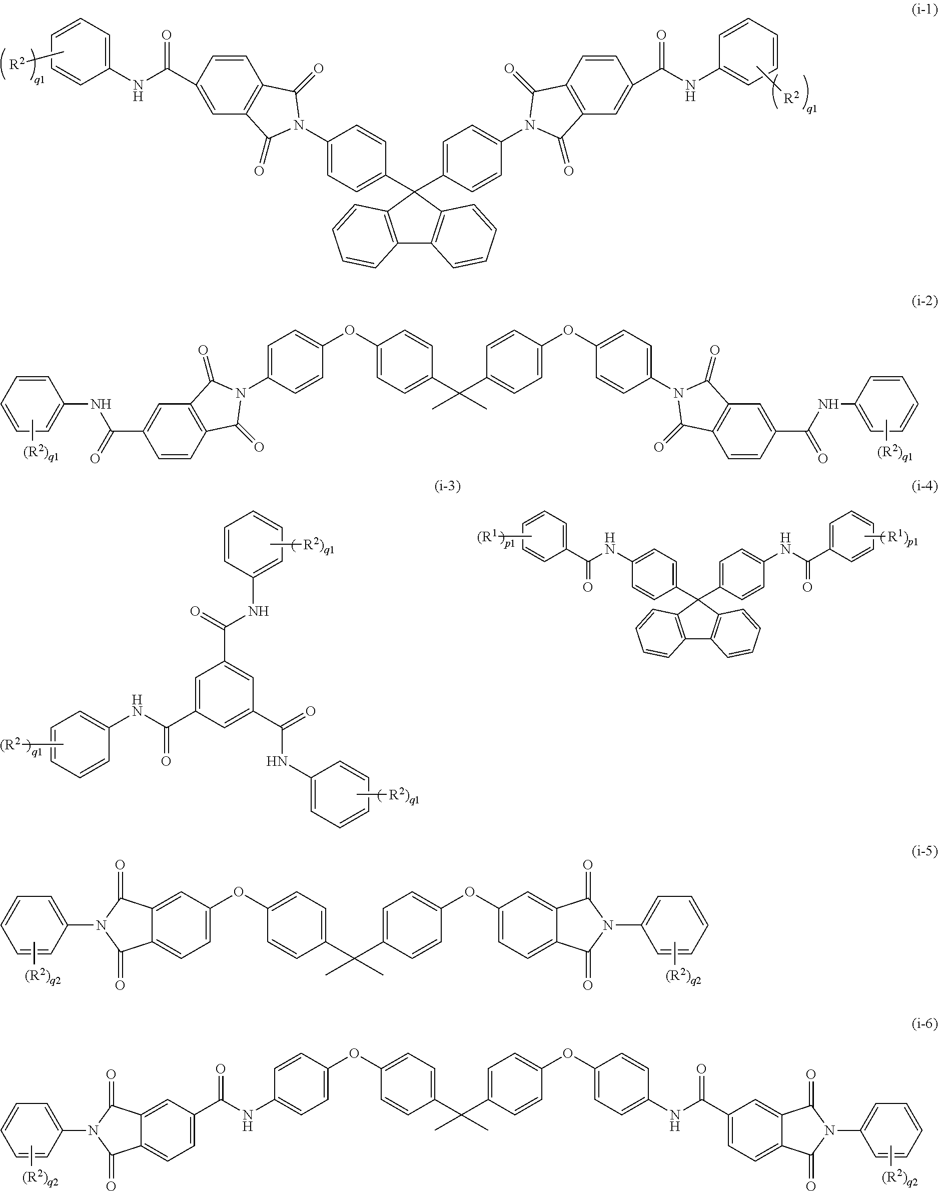

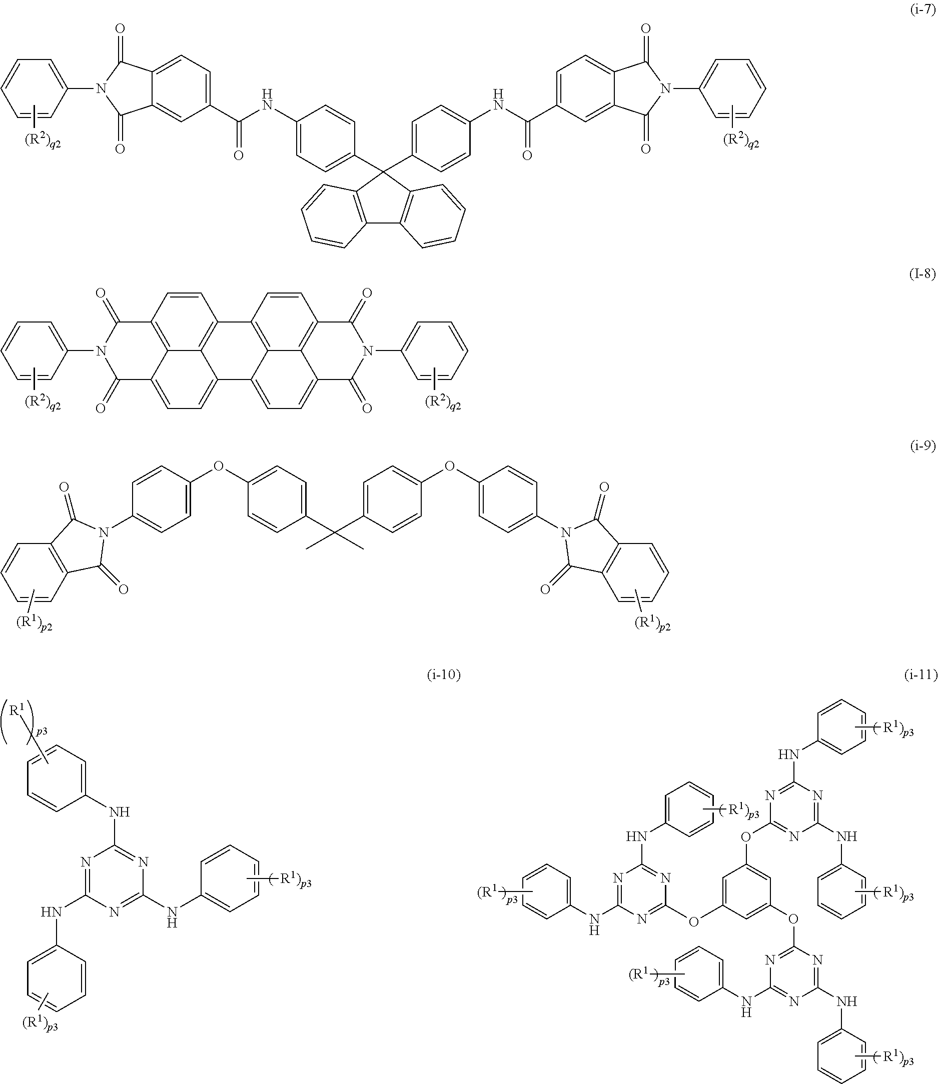

In the above formulae (i-1) to (i-11), R¹, R², p1, q1, p2, q2 and p3 are as defined in the above formulae (1-1) to (1-3).

The lower limit of a molecular weight of the compound (A) is preferably 300, more preferably 400, and still more preferably 500. The upper limit of the molecular weight is preferably 3,000, more preferably 2,000, and still more preferably 1,000. When the molecular weight of the compound (A) falls within the above range, flatness of the resist underlayer film can be further improved. The compound (A) may be used either alone of one type, or in a combination of two or more types thereof In a case in which two or more types of the compounds (A) are used, the molecular weight of the compound (A) as referred to means a number average molecular weight.

### Synthesis Method of Compound (A)

The compound (A) may be synthesized by, for example, allowing an aromatic carboxylic acid halide such as 4-ethynylbenzoyl chloride or benzenetricarbonyl trichloride; an aromatic carboxylic anhydride such as trimellitic anhydride chloride, 4,4′-(4,4′-isopropylidenediphenoxy)diphthalic anhydride, 4-phenylethynylcarbonyl phthalic anhydride, or 3,4,9,10-perylenetetracarboxylic dianhydride; a halogenated aromatic compound such as trichlorotriazine; or the like to react with an aromatic amine compound such as 3-ethynylaniline, 9,9-bis(4-aminophenyl)fluorene, 2,2-bis[4(4-aminophenoxy)phenyl]propane, 4-aminobenzyl alcohol, 4-dimethylaminoaniline, 3,4-methylenedioxyaniline, or 5-amino-2-naphthol, in a solvent such as N,N-dimethylacetamide, diisopropylethylamine, toluene, or tetrahydrofuran to form the structure of the group (I).

The upper limit of a percentage content of hydrogen atoms in the compound (A) is preferably 6.5% by mass, more preferably 6.0% by mass, still more preferably 5.0% by mass, and particularly preferably 4.0% by mass. The lower limit of the percentage content of hydrogen atoms is, for example, 0.1% by mass. When the percentage content of hydrogen atoms in the compound (A) falls within the above range, etching resistance of the resist underlayer film can be further improved.

The lower limit of a percentage content of the compound (A) with respect to total components of the composition other than the solvent (B) is preferably 50% by mass, more preferably 70% by mass, and still more preferably 85% by mass. The upper limit of the content is, for example, 100% by mass.

The lower limit of a percentage content of the compound (A) in the composition is preferably 1% by mass, more preferably 3% by mass, and still more preferably 5% by mass. The upper limit of the percentage content is preferably 50% by mass, more preferably 30% by mass, and still more preferably 15% by mass.

### (B) Solvent

The solvent (B) is not particularly limited as long as it can dissolve or disperse the compound (A), and the optional component(s) which may be contained as needed.

The solvent (B) is exemplified by an alcohol solvent, a ketone solvent, an ether solvent, an ester solvent, a nitrogen-containing solvent, and the like. The solvent (B) may be used alone of one type, or two or more types thereof may be used in combination.

Examples of the alcohol solvent include: monohydric alcohol solvents such as methanol, ethanol and n-propanol; polyhydric alcohol solvents such as ethylene glycol and 1,2-propylene glycol; and the like.

Examples of the ketone solvent include: chain ketone solvents such as methyl ethyl ketone and methyl isobutyl ketone; cyclic ketone solvents such as cyclohexanone; and the like.

Examples of the ether solvent include: polyhydric alcohol ether solvents, e.g., chain ether solvents such as n-butyl ether, and cyclic ether solvents such as tetrahydrofuran; polyhydric alcohol partial ether solvents such as diethylene glycol monomethyl ether; and the like.

Examples of the ester solvent include: carbonate solvents such as diethyl carbonate;

mono ester acetate solvents such as methyl acetate and ethyl acetate; lactone solvents such as γ-butyrolactone; polyhydric alcohol partial ether carboxylate solvents such as diethylene glycol monomethyl ether acetate and propylene glycol monomethyl ether acetate; ester lactate solvents such as methyl lactate and ethyl lactate; and the like.

Examples of the nitrogen-containing solvent include: chain nitrogen-containing solvents such as N,N-dimethylacetamide; cyclic nitrogen-containing solvents such as N-methylpyrrolidone; and the like.

The solvent (B) is preferably the ketone solvent and/or the ester solvent, more preferably the cyclic ketone solvent and/or the polyhydric alcohol partial ether carboxylate solvent, and still more preferably cyclohexanone and/or propylene glycol monomethyl ether acetate.

### Optional Components

The composition may contain as optional component(s), an acid generating agent, a crosslinking agent, a surfactant, an adhesion aid, and/or the like. These optional components may be used alone of one type, or two or more types thereof may be used in combination.

### Acid Generating Agent

The acid generating agent generates an acid by an action of heat and/or light to promote the crosslinking of molecules of the compound (A). When the composition contains the acid generating agent, a crosslinking reaction of molecules of the compound (A) is promoted and consequently the hardness of the resist underlayer film to be formed can be further increased. The acid generating agent may be used alone of one type, or two or more types thereof may be used in combination.

The acid generating agent is exemplified by an onium salt compound, an N-sulfonyloxyimide compound, and the like.

### Crosslinking Agent

The crosslinking agent forms crosslinking bonds between components such as the compound (A) in the composition, or forms cross-linked structures by its own molecules, through an action of heat and/or an acid. When the composition contains the crosslinking agent, the hardness of the resist underlayer film to be formed can be increased. The crosslinking agent may be used alone of one type, or two or more types thereof may be used in combination.

The crosslinking agent is exemplified by a polyfunctional (meth)acrylate compound, an epoxy compound, a hydroxymethyl group-substituted phenol compound, an alkoxyalkyl group-containing phenol compound, a compound having an alkoxyalkylated amino group, an aromatic ring-containing compound having a carbon-carbon triple bond-containing group, and the like.

### Preparation Procedure of Composition

The composition may be prepared, for example, by mixing the compound (A), the solvent (B), and as needed, the optional component(s) in a certain ratio, preferably followed by filtering a thus resulting mixture through a membrane filter, etc. of no greater than 0.1 μm. The lower limit of a concentration of the composition is preferably 0.1% by mass, more preferably 1% by mass, still more preferably 3% by mass, and particularly preferably 5% by mass. The upper limit of the concentration is preferably 50% by mass, more preferably 30% by mass, still more preferably 20% by mass, and particularly preferably 15% by mass. The concentration is a value (% by mass) determined by: baking 0.5 g of the composition at 250° C. for 30 min; measuring a mass of a residue of the composition; and dividing this resultant mass by the mass of the composition.

### Resist Underlayer Film

The resist underlayer film of the embodiment of the invention is formed from the composition of the embodiment of the present invention. Since the resist underlayer film is formed from the composition described above, the resist underlayer film is superior in etching resistance, heat resistance, flatness and the inhibitory property on film defects.

### Resist Underlayer Film-Forming Method

The resist underlayer film-forming method includes: a step of applying the composition of the embodiment of the invention directly or indirectly on at least an upper face of a substrate (hereinafter, may be also referred to as “applying step”).

Since the composition described above is used in the resist underlayer film-forming method, a resist underlayer film that is superior in etching resistance, heat resistance, flatness and an inhibitory property on film defects can be easily and reliably formed. The applying step will be described below.

### Applying Step

In this step, the composition of the embodiment of the invention is applied directly or indirectly on at least an upper face of a substrate to form a resist underlayer film.

Examples of the substrate include a silicon wafer, a wafer coated with aluminum, and the like. The applying procedure of the composition is not particularly limited, and for example, an appropriate procedure such as spin coating, cast coating or roll coating may be employed.

It is preferred that the coating film formed by the applying is heated.

Heating of the coating film is typically carried out in an ambient air, but may be carried out in a nitrogen atmosphere. A heating temperature is, for example, no less than 200° C. and no greater than 600° C. A heating time period is, for example, no less than 15 sec and no greater than 1,200 sec.

The coating film may be preheated at a temperature of no less than 60° C. and no greater than 150° C. before being heated at a temperature of no less than 200° C. and no greater than 600° C. The lower limit of the heating time period in the preheating is preferably 10 sec, and more preferably 30 sec. The upper limit of the heating time period is preferably 300 sec, and more preferably 180 sec.

It is to be noted that according to the resist underlayer film-forming method of the embodiment of the invention, in the case of heating the coating film to form the resist underlayer film, provided that the composition contains the acid generating agent and the acid generating agent is a radiation-sensitive acid generating agent, it is also possible to form the resist underlayer film by hardening the film through a combination of an exposure and heating. The radioactive ray used for the exposure may be appropriately selected from: electromagnetic waves such as visible rays, ultraviolet rays, far ultraviolet rays, X-rays and g-rays; and particle rays such as electron beams, molecular beams and ion beams in accordance with the type of the acid generating agent.

The lower limit of the average thickness of the resist underlayer film to be formed is preferably 30 nm, more preferably 50 nm, and still more preferably 100 nm. The upper limit of the average thickness is preferably 3,000 nm, more preferably 2,000 nm, and still more preferably 500 nm.

### Patterned Substrate-Producing Method

The patterned substrate-producing method of the embodiment of the present invention includes: a step of applying the composition of the embodiment of the invention directly or indirectly on at least an upper face of a substrate (hereinafter, may be also referred to as “applying step”); a step of forming a resist pattern on an upper face side of a resist underlayer film formed by the applying step (hereinafter, may be also referred to as “resist pattern-forming step”); and a step of carrying out etching using the resist pattern as a mask (hereinafter, may be also referred to as “etching step”).

According to the patterned substrate-producing method, use of the aforementioned resist underlayer film that is superior in etching resistance, heat resistance, flatness and an inhibitory property on film defects enables a favorable patterned substrate having a favorable pattern configuration to be obtained.

The patterned substrate-producing method may also include, as needed, a step of forming a silicon-containing film on an upper face side of the resist underlayer film formed by the applying step (hereinafter, may be also referred to as “silicon-containing film-forming step”). Hereinafter, each step will be described.

### Applying Step

In this step, the composition of the embodiment of the invention is applied directly or indirectly on at least an upper face of a substrate to form a resist underlayer film. This step is similar to the applying step in the resist underlayer film-forming method described above.

### Silicon-Containing Film-Forming Step

In this step, a silicon-containing film is formed on an upper face side of the resist underlayer film formed by the applying step.

The silicon-containing film may be formed by, for example: applying a composition for silicon-containing film formation on an upper face side of the resist underlayer film to form a coating film; and hardening the coating film typically by subjecting the coating film to an exposure and/or heating. As a commercially available product of the composition for silicon-containing film formation, for example, “NFC SOG01”, “NFC SOG04”, or “NFC SOG080” (all available from JSR Corporation), or the like may be used. Furthermore, the silicon-containing film may be formed by a CVD process, a PVD process, or the like. The CVD process is exemplified by a plasma-enhanced CVD procedure, a low-pressure CVD procedure, an epitaxial growth procedure, and the like. The PVD process is exemplified by a sputtering procedure, an evaporation procedure, and the like.

Examples of the radioactive ray for use in the exposure include: electromagnetic waves such as visible rays, ultraviolet rays, far ultraviolet rays, X-rays and γ-rays; particle rays such as electron beams, molecular beams and ion beams; and the like.

The lower limit of a temperature for heating the coating film is preferably 90° C., more preferably 150° C., and still more preferably 200° C. The upper limit of the temperature is preferably 550° C., more preferably 450° C., and still more preferably 300° C. The lower limit of an average thickness of the silicon-containing film to be formed is preferably 1 nm, more preferably 10 nm, and still more preferably 20 nm. The upper limit of the average thickness is preferably 20,000 nm, more preferably 1,000 nm, and still more preferably 100 nm.

### Resist Pattern-Forming Step

In this step, a resist pattern is formed on an upper face side of the resist underlayer film described above. In the case in which the silicon-containing film is formed in the silicon-containing film-forming step, a resist pattern is formed on an upper face side of the silicon-containing film. This step may be carried out by, for example, using a resist composition, or the like.

When the resist composition is used, specifically, the resist film is formed by applying the resist composition by a spin-coating procedure or the like such that a resultant resist film has a predetermined thickness, and thereafter subjecting the resist composition to prebaking to evaporate away the solvent in the coating film.

Examples of the resist composition include a chemically amplified positive or negative resist composition that contains a radiation-sensitive acid generating agent; a positive resist composition containing an alkali-soluble resin and a quinone diazide-based photosensitizing agent; a negative resist composition containing an alkali-soluble resin and a crosslinking agent; and the like.

Next, the resist film formed is exposed by selective irradiation with a radioactive ray. The radioactive ray used in the exposure may be appropriately selected from: electromagnetic waves such as visible rays, ultraviolet rays, far ultraviolet rays, X-rays and g-rays; and particle rays such as electron beams, molecular beams and ion beams in accordance with the type of the radiation-sensitive acid generating agent to be used in the resist composition. Among these, far ultraviolet rays are preferred, a KrF excimer laser beam (wavelength: 248 nm), an ArF excimer laser beam (wavelength: 193 nm), an F2 excimer laser beam (wavelength: 157 nm), a Kr₂ excimer laser beam (wavelength: 147 nm), an ArKr excimer laser beam (wavelength: 134 nm), or an extreme ultraviolet ray (EUV; wavelength: 13.5 nm, etc.) is more preferred, and a KrF excimer laser beam, an ArF excimer laser beam, or EUV is still more preferred.

Post-baking may be carried out after the exposure for the purpose of improving resolution, pattern profile, developability, and the like.

Next, the resist film exposed is developed with a developer solution to form a resist pattern. The development may be either a development with an alkali or a development with an organic solvent. In the case of the development with an alkali, examples of the developer solution include basic aqueous solutions of tetramethyl ammonium hydroxide (TMAH), tetraethyl ammonium hydroxide, or the like. Alternatively, in the case of the development with an organic solvent, examples of the developer solution include organic solvents such as n-butyl acetate, iso-butyl acetate, sec-butyl acetate, and amyl acetate, and the like.

A predetermined resist pattern is formed by the development with the developer solution, followed by washing and drying.

In carrying out the resist pattern-forming step, aside from using the resist composition described above, another process may be employed; for example, a nanoimprinting procedure may be adopted, or a directed self-assembling composition may be also used.

### Etching Step

In this step, etching is carried out with the aforementioned resist pattern as a mask to form a pattern on the substrate. The etching may be conducted once or multiple times. In other words, the etching may be conducted sequentially with patterns obtained by the etching as masks. In the case in which the etching is conducted multiple times, the silicon-containing film, the resist underlayer film, and the substrate are subjected to the etching sequentially in this order. The etching step may be exemplified by dry etching, wet etching, and the like. After the etching, the substrate having a predetermined pattern can be obtained.

The dry etching may be carried out by using, for example, a known dry etching apparatus. An etching gas used for the dry etching may be appropriately selected depending on the mask pattern as well as on the element composition and the like of the film to be etched. Examples of the etching gas which may be used include: fluorine-based gasses such as CHF₃, CF₄, C₂F₆, C₃F₈ and SF₆; chlorine-based gasses such as Cl₂ and BCl₃; oxygen-based gasses such as O₂, O₃ and H₂O; reductive gasses such as H₂, NH₃, CO, CO₂, CH₄, C₂H₂, C₂H₄, C₂H₆, C₃H₄, C₃H₆, C₃H₈, HF, HI, HBr, HCl, NO, NH₃ and BCl₃; inert gasses such as He, N₂ and Ar; and the like. These gasses may be used as a mixture. In the case in which the substrate is etched by using the pattern of the resist underlayer film as a mask, the fluorine-based gas is typically used.

### EXAMPLES

Hereinafter, the embodiment of the present invention will be explained in more detail by way of Examples, but the present invention is not in any way limited to these Examples. Measuring methods for various types of physical properties are shown below.

### Average Thickness of Film

The average thickness of the film was measured by using a spectroscopic ellipsometer (“M2000D” available from J. A. WOOLLAM Co.).

### Synthesis of Compound (A)

Compounds represented by the following formulae (A-1) to (A-16) (hereinafter, may be also referred to as “compounds (A-1) to (A-16)”) were synthesized in accordance with the following procedure.

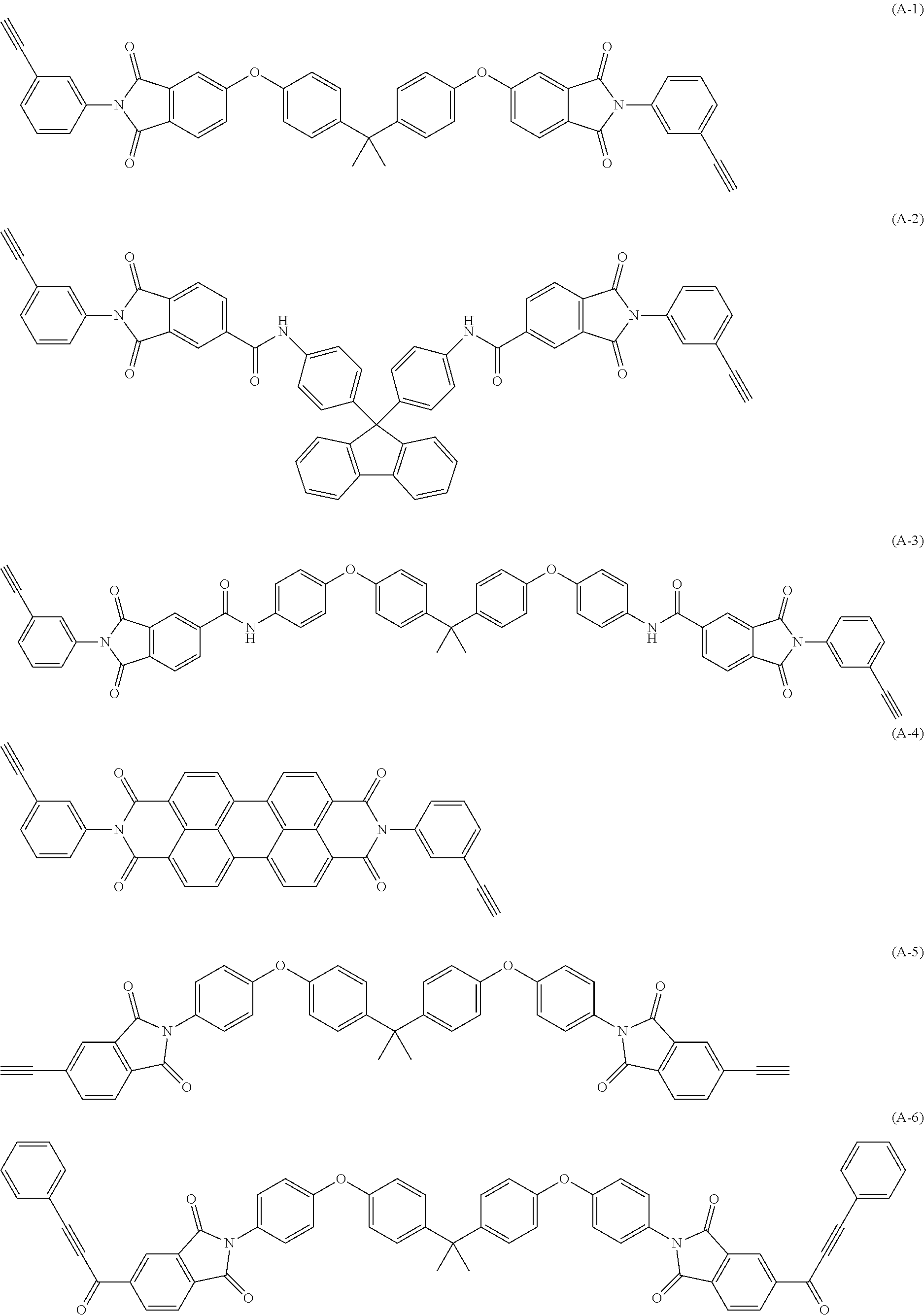

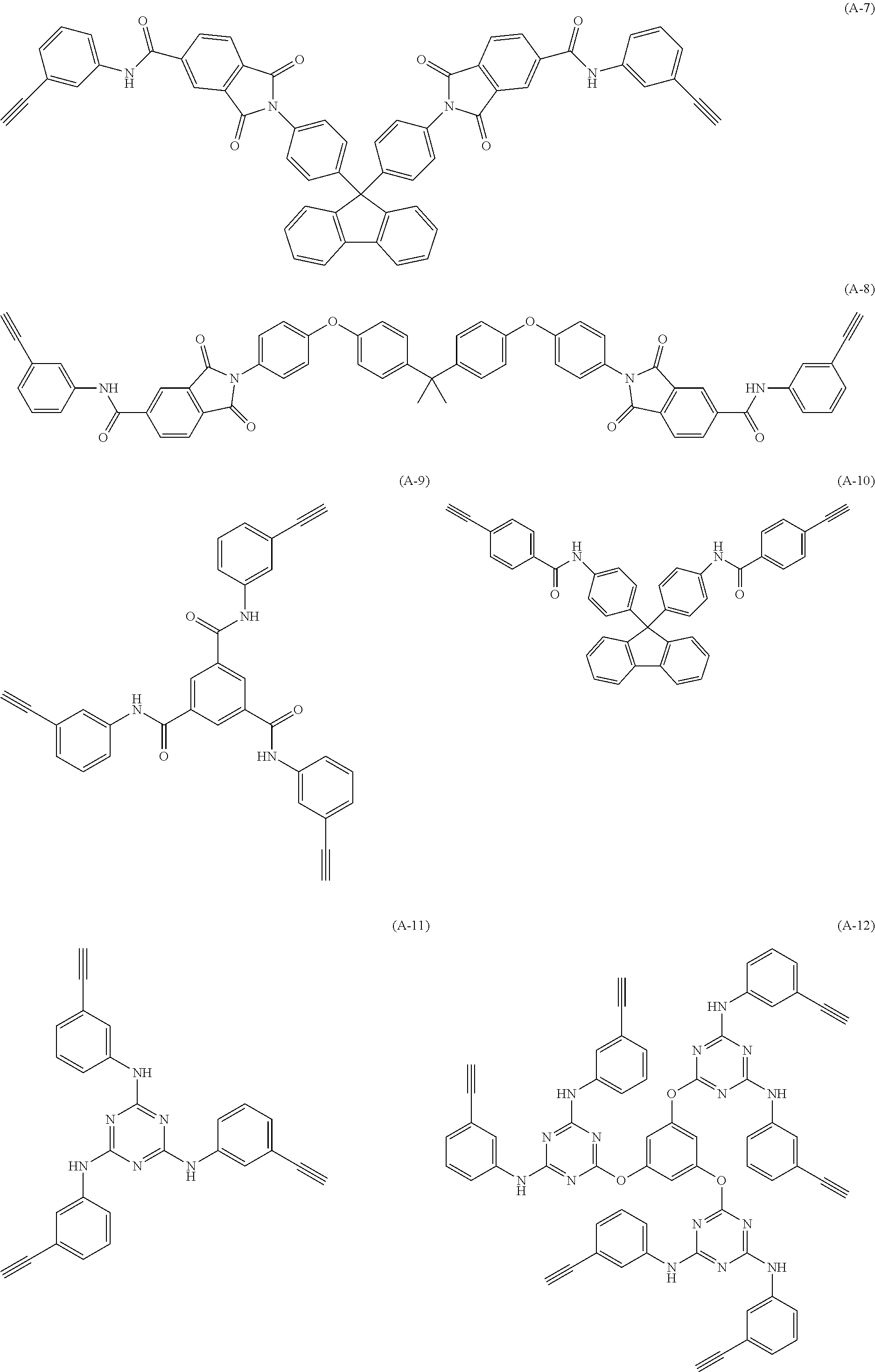

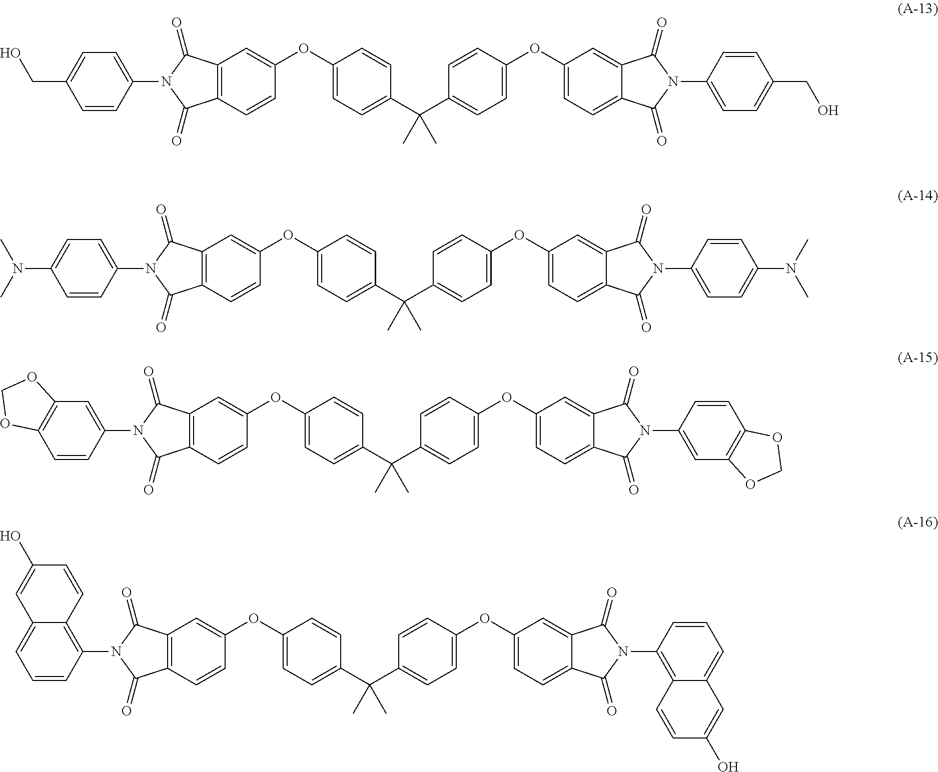

### Synthesis Example 1-1

The compound (A-1) was obtained by: adding in a nitrogen atmosphere 20.0 g of 4,4′-(4,4′-isopropylidenediphenoxy)diphthalic anhydride, 9.0 g of 3-ethynylaniline, and 120.0 g of N,N-dimethylacetamide into a reaction vessel; and allowing for a reaction at 150° C. for 3 hrs.

### Synthesis Example 1-2

Into a reaction vessel, 15.0 g of trimellitic anhydride chloride, 9.9 g of 9,9-bis(4-aminophenyl)fluorene, and 65.0 g of N,N-dimethylacetamide were added in a nitrogen atmosphere, and a reaction was allowed at 0° C. for 3 hrs. Thereafter, 15.0 g of 3-ethynylaniline was further added at room temperature and a reaction was allowed at 150° C. for 3 hrs to give the compound (A-2).

### Synthesis Example 1-3

Into a reaction vessel, 15.0 g of trimellitic anhydride chloride, 11.7 g of 2,2-bis[4-(4-aminophenoxy)phenyl]propane, and 65.0 g of N,N-dimethylacetamide were added in a nitrogen atmosphere, and a reaction was allowed at 0° C. for 3 hrs. Thereafter, 15.0 g of 3-ethynylaniline was further added at room temperature and a reaction was allowed at 150° C. for 3 hrs to give the compound (A-3).

### Synthesis Example 1-4

The compound (A-4) was obtained by: adding in a nitrogen atmosphere 20.0 g of 3,4,9,10-perylenetetracarboxylic dianhydride, 6.0 g of 3-ethynylaniline, and 120.0 g of N,N-dimethylacetamide into a reaction vessel; and allowing for a reaction at 150° C. for 3 hrs.

### Synthesis Example 1-5

The compound (A-5) was obtained by: adding in a nitrogen atmosphere 20.0 g of 2,2-bis[4-(4-aminophenoxy)phenyl]propane, 16.7 g of 4-ethynyl phthalic anhydride, and 120.0 g of N,N-dimethylacetamide into a reaction vessel; and allowing for a reaction at 150° C. for 3 hrs.

### Synthesis Example 1-6

The compound (A-6) was obtained by: adding in a nitrogen atmosphere 20.0 g of 2,2-bis[4-(4-aminophenoxy)phenyl]propane, 24.2 g of 4-phenylethynylcarbonyl phthalic anhydride, and 120.0 g of N,N-dimethylacetamide into a reaction vessel; and allowing for a reaction at 150° C. for 3 hrs.

### Synthesis Example 1-7

Into a reaction vessel, 15.0 g of trimellitic anhydride chloride, 8.4 g of 3-ethynylaniline, and 65.0 g of N,N-dimethylacetamide were added in a nitrogen atmosphere, and a reaction was allowed at 0° C. for 3 hrs. Thereafter, 12.4 g of 9,9-bis(4-aminophenyl)fluorene was further added at room temperature and a reaction was allowed at 150° C. for 3 hrs to give the compound (A-7).

### Synthesis Example 1-8

Into a reaction vessel, 15.0 g of trimellitic anhydride chloride, 8.4 g of 3-ethynylaniline, and 65.0 g of N,N-dimethylacetamide were added in a nitrogen atmosphere, and a reaction was allowed at 0° C. for 3 hrs. Thereafter, 14.6 g of 2,2-bis[4-(4-aminophenoxy)phenyl]propane was further added at room temperature and a reaction was allowed at 150° C. for 3 hrs to give the compound (A-8).

### Synthesis Example 1-9

Into a reaction vessel, 15.0 g of 1,3,5-benzenetricarbonyl trichloride, 21.8 g of 3-ethynylaniline, and 184.2 g of N,N-dimethylacetamide were added in a nitrogen atmosphere, and a reaction was allowed at 0° C. for 1 hour. Thereafter, the reaction was allowed at room temperature for 3 hrs to give the compound (A-9).

### Synthesis Example 1-10

Into a reaction vessel, 15.0 g of 9,9-bis(4-aminophenyl)fluorene, 14.2 g of 4-ethynyl benzoyl chloride, and 116.7 g of N,N-dimethylacetamide were added in a nitrogen atmosphere, and a reaction was allowed at 0° C. for 1 hour. Thereafter, the reaction was allowed at room temperature for 3 hrs to give the compound (A-10).

### Synthesis Example 1-11

Into a reaction vessel, 15.0 g of trichlorotriazine, 28.6 g of 3-ethynylaniline, and 130.8 g of toluene were added in a nitrogen atmosphere, and a reaction was allowed at 0° C. for 1 hour. Thereafter, the reaction was allowed at 110° C. for 3 hrs to give the compound (A-11).

### Synthesis Example 1-12

Into a reaction vessel, 65.8 g of trichlorotriazine and 329.0 g tetrahydrofuran were added in a nitrogen atmosphere, and then a solution prepared by dissolving 15.0 g of phloroglucinol and 46.1 g of diisopropylethylamine in 300.0 g of tetrahydrofuran was added dropwise thereto at 0° C. over 1 hour. Subsequently, a reaction was allowed at room temperature for 2 hrs. Thereafter, 97.5 g of 3-ethynylaniline and 107.6 g of diisopropylethylamine were further added and a reaction was allowed at 65° C. for 3 hrs to give the compound (A-12).

### Synthesis Example 1-13

The compound (A-13) was obtained by: adding in a nitrogen atmosphere 15.0 g of 4,4′-(4,4′-isopropylidenediphenoxy)diphthalic anhydride, 7.1 g of 4-aminobenzyl alcohol, and 90.0 g of N,N-dimethylacetamide into a reaction vessel; and allowing for a reaction at 150° C. for 3 hrs.

### Synthesis Example 1-14

The compound (A-14) was obtained by: adding in a nitrogen atmosphere 15.0 g of 4,4′-(4,4′-isopropylidenediphenoxy)diphthalic anhydride, 7.9 g of 4-dimethylaminoaniline, and 90.0 g of N,N-dimethylacetamide into a reaction vessel; and allowing for a reaction at 150° C. for 3 hrs.

### Synthesis Example 1-15

The compound (A-15) was obtained by: adding in a nitrogen atmosphere 15.0 g of 4,4′-(4,4′-isopropylidenediphenoxy)diphthalic anhydride, 7.9 g of 3,4-methylenedioxyaniline, and 90.0 g of N,N-dimethylacetamide into a reaction vessel; and allowing for a reaction at 150° C. for 3 hrs.

### Synthesis Example 1-16

The compound (A-16) was obtained by: adding in a nitrogen atmosphere 15.0 g of 4,4′-(4,4′-isopropylidenediphenoxy)diphthalic anhydride, 9.2 g of 5-amino-2-naphthol, and 90.0 g of N,N-dimethylacetamide into a reaction vessel; and allowing for a reaction at 150° C. for 3 hrs.

### Synthesis Example 2-1

Into a reaction vessel, 250.0 g of m-cresol, 125.0 g of 37% by mass formalin, and 2 g of anhydrous oxalic acid were added in a nitrogen atmosphere, and a reaction was allowed at 100° C. for 3 hrs and then at 180° C. for 1 hour. Subsequently, an unreacted monomer was eliminated under a reduced pressure to give a resin represented by the following formula (a-1). A weight average molecular weight (Mw) of the resin (a-1) thus obtained was determined to be 11,000 as a result of a measurement by gel permeation chromatography (detector: differential refractometer) using GPC columns available from Tosoh Corporation (“G2000 HXL”×2 and “G3000 HXL”×1), under analytical conditions involving a flow rate of 1.0 mL/min, an elution solvent of tetrahydrofuran, and a column temperature of 40° C., with mono-dispersed polystyrene as a standard.

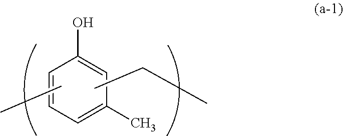

### Preparation of Composition for Resist Underlayer Film Formation

The compound (A), the solvent (B), an acid generating agent (hereinafter, may be also referred to as “(C) acid generating agent” or “acid generating agent (C)”), and a crosslinking agent (hereinafter, may be also referred to as “(D) crosslinking agent” or “crosslinking agent (D)”) used in preparation of the compositions for resist underlayer film formation are as presented below.

### (A) Compound

Examples: the compounds (A-1) to (A-16) synthesized as described above

Comparative Example: the resin (a-1) synthesized as described above

### (B) Solvent

B-1: cyclohexanone

B-2: propylene glycol monomethyl ether acetate

### (C) Acid Generating Agent

C-1: bis(4-t-butylphenyl)iodonium nonafluoro-n-butanesulfonate (a compound represented by the following formula (C-1))

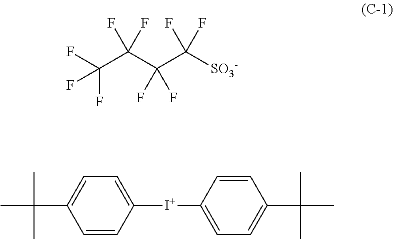

### (D) Crosslinking Agent

D-1: 1,3,4,6-tetrakis(methoxymethyl)glycoluril (a compound represented by the following formula (D-1))

D-2: a compound represented by the following formula (D-2)

D-3: a compound represented by the following formula (D-3)

D-4: a compound represented by the following formula (D-4)

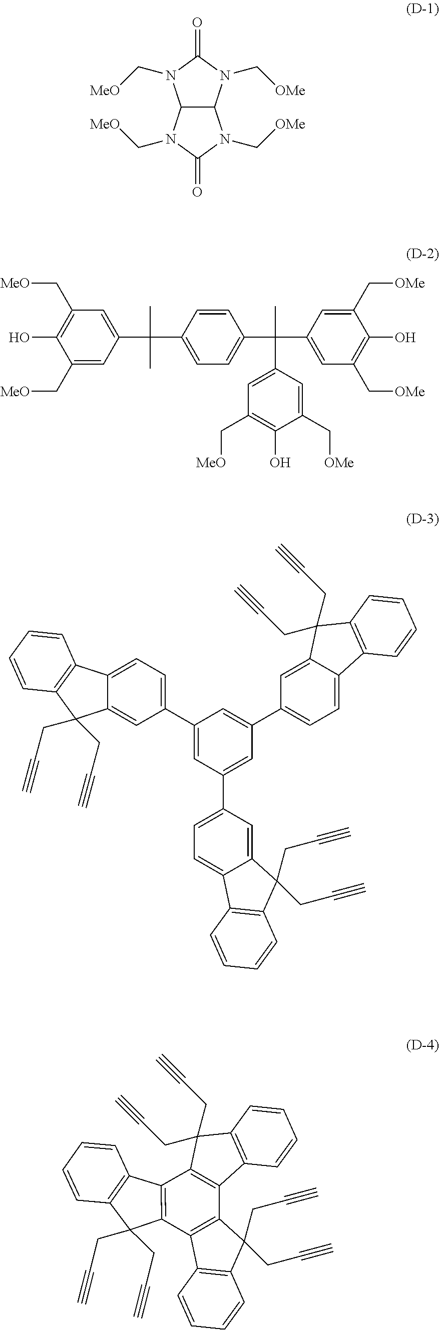

### Example 1-1

Ten parts by mass of (A-1) as the compound (A) were dissolved in 90 parts by mass of (B-1) as the solvent (B). A solution thus obtained was filtered through a membrane filter having a pore size of 0.1 μm to prepare a composition for resist underlayer film formation (J-1).

### Examples 1-2 to 1-20 and Comparative Example 1-1

Compositions for resist underlayer film formation (J-2) to (J-20) and (CJ-1) were prepared by a similar operation to that of Example 1-1 except that for each component, the type and content shown in Table 1 were used. In Table 1, “-” indicates that a corresponding component was not used.

TABLE 1

| | | | | | | (C) Acid | | (D) Crosslinking | |
|---|---|---|---|---|---|---|---|---|---|
| | Composition | (A) Compound | | (B) Solvent | | generating agent | | agent | |
| | for resist | | content | | content | | content | | content |
| | underlayer | | (parts by | | (parts by | | (parts by | | (parts by |
| | film formation | Type | mass) | type | mass) | type | mass) | type | mass) |
| Example 1-1 | J-1 | A-1 | 10 | B-1 | 90 | — | — | — | — |
| Example 1-2 | J-2 | A-2 | 10 | B-2 | 90 | — | — | — | — |
| Example 1-3 | J-3 | A-3 | 10 | B-1 | 90 | — | — | — | — |
| Example 1-4 | J-4 | A-4 | 10 | B-1 | 90 | — | — | — | — |
| Example 1-5 | J-5 | A-5 | 10 | B-1 | 90 | — | — | — | — |
| Example 1-6 | J-6 | A-6 | 10 | B-1 | 90 | — | — | — | — |
| Example 1-7 | J-7 | A-7 | 10 | B-1 | 90 | — | — | — | — |
| Example 1-8 | J-8 | A-8 | 10 | B-1 | 90 | — | — | — | — |
| Example 1-9 | J-9 | A-9 | 10 | B-1 | 90 | — | — | — | — |
| Example 1-10 | J-10 | A-10 | 10 | B-1 | 90 | — | — | — | — |
| Example 1-11 | J-11 | A-11 | 10 | B-2 | 90 | — | — | — | — |
| Example 1-12 | J-12 | A-12 | 10 | B-2 | 90 | — | — | — | — |
| Example 1-13 | J-13 | A-13 | 10 | B-1 | 90 | — | — | — | — |
| Example 1-14 | J-14 | A-14 | 10 | B-1 | 90 | — | — | — | — |
| Example 1-15 | J-15 | A-15 | 10 | B-1 | 90 | — | — | — | — |
| Example 1-16 | J-16 | A-16 | 10 | B-1 | 90 | — | — | — | — |
| Example 1-17 | J-17 | A-1 | 10 | B-1 | 90 | C-1 | 0.5 | D-1 | 1 |
| Example 1-18 | J-18 | A-1 | 10 | B-1 | 90 | C-1 | 0.5 | D-2 | 1 |
| Example 1-19 | J-19 | A-1 | 10 | B-1 | 90 | — | — | D-3 | 5 |
| Example 1-20 | J-20 | A-1 | 10 | B-1 | 90 | — | — | D-4 | 5 |
| Comparative | CJ-1 | a-1 | 10 | B-1 | 90 | — | — | — | — |
| Example 1-1 | | | | | | | | | |

### Formation of Resist Underlayer Film

### Examples 2-1 to 2-20 and Comparative Example 2-1

Each composition for resist underlayer film formation prepared as described above was applied on a silicon wafer (substrate) with a spin coater (“CLEAN TRACK ACT12” available from Tokyo Electron Limited) by way of a spin-coating procedure. Next, heating (baking) in an ambient air atmosphere at the heating temperature (° C.) for the heating time period (sec) shown in Table 2 below was followed by cooling at 23° C. for 60 sec to form a resist underlayer film having an average thickness of 200 nm. Accordingly, a resist underlayer film-attached substrate having been provided with the resist underlayer film thereon was obtained.

### Evaluations

By using the compositions for resist underlayer film formation obtained and the resist underlayer film-attached substrates obtained, the following evaluations were each made according to the following procedures. The results of the evaluations are shown together in Table 2 below. In Table 2, “-” indicates hat the corresponding item is a standard for evaluation of etching resistance.

### Etching Resistance

The resist underlayer film of the resist underlayer film-attached substrate obtained as described above was treated in an etching apparatus (“TACTRAS” available from Tokyo Electron Limited) under conditions involving: CF₄/Ar=110/440 sccm, PRESS.=30 MT, HF RF (radiofrequency power for plasma production)=500 W, LF RF (radiofrequency power for bias)=3,000 W, DCS=−150 V, RDC (flow rate percentage at gas center)=50%, and 30 sec. An etching rate (nm/min) was calculated based on the average thickness of the resist underlayer film before the treatment and the average thickness of the resist underlayer film after the treatment, and a ratio with respect to Comparative Example 2-1 was determined as a marker for etching resistance evaluation. The etching resistance was evaluated to be: “A” (favorable) in a case in which the ratio was no less than 0.98 and less than 1.00; and “B” (unfavorable) in a case in which the ratio was no less than 1.00.

### Heat Resistance

The composition for resist underlayer film formation prepared as described above was applied on a silicon wafer having a diameter of 8 inches by a spin coating procedure and baked in an ambient air atmosphere at 250° C. for 60 sec to form a resist underlayer film, whereby a resist underlayer film-attached substrate was obtained. Next, the resist underlayer film of this resist underlayer film-attached substrate was scraped to collect the powder, and the powder of the resist underlayer film was placed into a container used for measurement by a TG-DTA apparatus (“TG-DTA 2000SR”, available from NETZSCH), and a mass of the powder prior to heating was measured. The powder was then heated to 400° C. in the TG-DTA apparatus in a nitrogen atmosphere with a rate of temperature rise of 10° C./min, and the mass of the powder at 400° C. was measured. The mass loss rate (%) was obtained by the following equation and defined as a marker of heat resistance.

M_{L}={(m1−m2)/m1}×100

In the above equation, M_{L} represents the mass loss rate (%); m1 represents the mass prior to heating (mg); and m2 represents the mass at 400° C. (mg).

A lower mass loss rate of the powder used as the sample indicates that the heat resistance is more favorable, due to less generation of sublimated matter and resist underlayer film degradation products during the heating of the resist underlayer film. In other words, the lower mass loss rate indicates higher heat resistance. The heat resistance was evaluated to be: “A” (extremely favorable) in a case in which the mass loss rate was less than 5%; “B” (favorable) in a case in which the mass loss rate was no less than 5% and less than 10%; and “C” (unfavorable) in a case in which the mass loss rate was no less than 10%.

### Flatness

Each of the compositions for resist underlayer film formation prepared as described above was applied by a spin-coating procedure using a spin coater (“CLEAN TRACK ACT-12” available from Tokyo Electron Limited), on a silicon substrate 1 provided with a trench pattern having a depth of 100 nm and a groove width of 10 μm formed thereon, as shown in the Figure. A rotational speed for the spin coating was the same as that in the case of forming the resist underlayer film having the average thickness of 200 nm in “Formation of Resist Underlayer Film,” described above. Subsequently, by heating (baking) in an ambient air atmosphere at a heating temperature (° C.) for a heating time period (sec) shown in Table 2 below, a resist underlayer film 2 was formed having an average thickness of 200 nm at parts having no trench provided. Accordingly, a resist underlayer film-attached silicon substrate, the silicon substrate being covered by the resist underlayer film, was obtained.

A cross-sectional shape of the resist underlayer film-attached silicon substrate was observed by using a scanning electron microscope (“S-4800” available from Hitachi High-Technologies Corporation), and the difference (ΔFT) between a height at a center portion “b” of the trench pattern of the resist underlayer film and a height at a position “a” 5 μtm away from the edge of the trench pattern, at which no trench pattern was provided, was defined as a marker of the flatness. The flatness was evaluated to be: “A” (favorable) in a case of ΔFT being less than 40 nm; “B” (somewhat favorable) in a case of ΔFT being no less than 40 nm and less than 60 nm; and “C” (unfavorable) in a case of ΔFT being no less than 60 nm. It is to be noted that the difference in heights shown in the Figure is exaggerated.

### Inhibitory Property on Film Defects

On the resist underlayer film-attached substrate obtained as described above, a composition for silicon-containing film formation (“NFC SOG080” available from JSR Corporation) was applied by a spin-coating procedure and then heated (baked) at 200° C. for 60 sec in an ambient air atmosphere to form a silicon-containing film having an average thickness of 50 nm, thereby giving a substrate provided with a silicon-containing film. After the substrate provided with the silicon-containing film thus obtained was further heated (baked) at 450° C. for 60 sec, the surface of the silicon-containing film was observed with an optical microscope. The inhibitory property on film defects was evaluated to be: “A” (favorable) in a case in which cracking or peeling of the silicon-containing film was not found; and “B” (unfavorable) in a case in which cracking or peeling of the silicon-containing film was found.

TABLE 2

| | Composition | Heating | | | | |
|---|---|---|---|---|---|---|
| | for resist | temperature/heating time | | | | |
| | underlayer | period in resist | | | | Inhibitory |
| | film | underlayer film | Etching | Heat | | property on |
| | formation | formation (° C./sec) | resistance | resistance | Flatness | film defects |
| Example 2-1 | J-1 | 350/60 | A | A | A | A |
| Example 2-2 | J-2 | 350/60 | A | B | A | A |
| Example 2-3 | J-3 | 350/60 | A | A | A | A |
| Example 2-4 | J-4 | 350/60 | A | A | A | A |
| Example 2-5 | J-5 | 350/60 | A | A | A | A |
| Example 2-6 | J-6 | 350/60 | A | A | A | A |
| Example 2-7 | J-7 | 350/60 | A | B | A | A |
| Example 2-8 | J-8 | 350/60 | A | A | A | A |
| Example 2-9 | J-9 | 350/60 | A | A | A | A |
| Example 2-10 | J-10 | 350/60 | A | A | A | A |
| Example 2-11 | J-11 | 350/60 | A | B | A | A |
| Example 2-12 | J-12 | 350/60 | A | B | A | A |
| Example 2-13 | J-13 | 350/60 | A | A | A | A |
| Example 2-14 | J-14 | 350/60 | A | A | A | A |
| Example 2-15 | J-15 | 350/60 | A | A | A | A |
| Example 2-16 | J-16 | 350/60 | A | A | A | A |
| Example 2-17 | J-17 | 350/60 | A | B | A | A |
| Example 2-18 | J-18 | 350/60 | A | B | A | A |
| Example 2-19 | J-19 | 350/60 | A | A | A | A |
| Example 2-20 | J-20 | 350/60 | A | A | A | A |
| Comparative | CJ-1 | 350/60 | — | C | C | B |
| Example 2-1 | | | | | | |

As is seen from the results shown in Table 2, the resist underlayer films formed from the compositions for resist underlayer film formation of the Examples were superior in all of the etching resistance, the heat resistance, the flatness, and the inhibitory property on film defects. To the contrary, the resist underlayer film formed from the composition for resist underlayer film formation of the Comparative Example was inferior in all of the etching resistance, the heat resistance, the flatness, and the inhibitory property on film defects.

The composition for resist underlayer film formation of the one embodiment of the present invention enables forming of a resist underlayer film that is superior in etching resistance, heat resistance, flatness and an inhibitory property on film defects. The resist underlayer film of the another embodiment of the present invention is superior in etching resistance, heat resistance, flatness and an inhibitory property on film defects. The resist underlayer film-forming method of the still another embodiment of the present invention enables a resist underlayer film that is superior in etching resistance, heat resistance, flatness and an inhibitory property on film defects to be easily and reliably formed. According to the patterned substrate-producing method of the yet another embodiment of the present invention, by using such a superior resist underlayer film, obtaining a favorable patterned substrate is enabled. Therefore, these can be suitably used for manufacture of semiconductor devices and the like, for which microfabrication is expected to progress further hereafter.

Obviously, numerous modifications and variations of the present invention are possible in light of the above teachings. It is therefore to be understood that within the scope of the appended claims, the invention may be practiced otherwise than as specifically described herein.

What is claimed is:

1. A composition comprising:
a compound comprising at least one group selected from the group consisting of a group represented by formula (1-1), a group represented by formula (1-2), and a group represented by formula (1-3), the compound having a molecular weight of no greater than 3,000; and
a solvent, 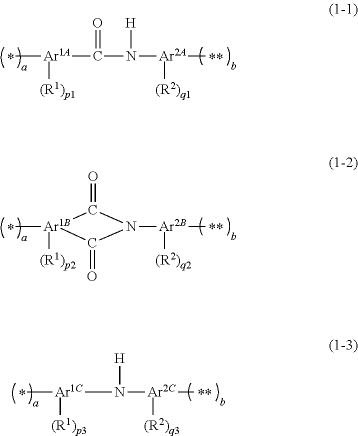
wherein,
in the formulae (1-1) to (1-3), * and ** each denote a site bonding to a part other than the group represented by the formulae (1-1) to (1-3) in the compound; and a and b are each independently an integer of 0 to 3, wherein in a case in which a is 0, b is no less than 1, and in a case in which a is no less than 1, b is 0,
in the formula (1-1), Ar^{1A} represents an aromatic carbocyclic group having 6 to 20 ring atoms and having a valency of (a+p1+1), or an aromatic heterocyclic group having 5 to 20 ring atoms and having a valency of (a+p1+1); R¹ represents a halogen atom, a hydroxy group, a nitro group, or a monovalent organic group having 1 to 20 carbon atoms; p1 is an integer of 0 to 11, wherein in a case in which p1 is no less than 2, a plurality of R¹s are identical or different; Ar^{2A} represents an aromatic carbocyclic group having 6 to 20 ring atoms and having a valency of (b+q1+1), or an aromatic heterocyclic group having 5 to 20 ring atoms and having a valency of (b+q1+1); R² represents a halogen atom, a hydroxy group, a nitro group, or a monovalent organic group having 1 to 20 carbon atoms; q1 is an integer of 0 to 11, wherein in a case in which q1 is no less than 2, a plurality of R²s are identical or different; a sum of p1 and a is no greater than 11; and a sum of q1 and b is no greater than 11,
in the formula (1-2), Ar^{1B} represents an aromatic carbocyclic group having 6 to 20 ring atoms and having a valency of (a+p2+2), or an aromatic heterocyclic group having 5 to 20 ring atoms and having a valency of (a+p2+2); R¹ represents a halogen atom, a hydroxy group, a nitro group, or a monovalent organic group having 1 to 20 carbon atoms; p2 is an integer of 0 to 10, wherein in a case in which p2 is no less than 2, a plurality of R¹s are identical or different; Ar^{2B} represents an aromatic carbocyclic group having 6 to 20 ring atoms and having a valency of (b+q2+1), or an aromatic heterocyclic group having 5 to 20 ring atoms and having a valency of (b+q2+1); q2 is an integer of 0 to 11, wherein in a case in which q2 is 1, R² represents a halogen atom, a hydroxy group, a nitro group, or a monovalent organic group having 1 to 20 carbon atoms, and in a case in which q2 is no less than 2, a plurality of R²s are identical or different, and each R² represents a halogen atom, a hydroxy group, a nitro group, or a monovalent organic group having 1 to 20 carbon atoms, and optionally two or more of the plurality of R²s taken together represent a ring structure having 4 to 20 ring atoms together with the atom chain to which the two or more of the plurality of R²s bond; a sum of p2 and a is no greater than 10; and a sum of q2 and b is no greater than 11, and
in the formula (1-3), Ar^{1C} represents an aromatic carbocyclic group having 6 to 20 ring atoms and having a valency of (a+p3+1), or an aromatic heterocyclic group having 5 to 20 ring atoms and having a valency of (a+p3+1); R¹ represents a halogen atom, a hydroxy group, a nitro group, or a monovalent organic group having 1 to 20 carbon atoms; p3 is an integer of 0 to 11, wherein in a case in which p3 is no less than 2, a plurality of R¹s are identical or different; Ar^{2C} represents an aromatic heterocyclic group having 5 to 20 ring atoms and having a valency of (b+q3+1); R² represents a halogen atom, a hydroxy group, a nitro group, or a monovalent organic group having 1 to 20 carbon atoms; q3 is an integer of 0 to 11, wherein in a case in which q3 is no less than 2, a plurality of R²s are identical or different; a sum of p3 and a is no greater than 11; and a sum of q3 and b is no greater than 11.

2. The composition according to claim 1, wherein the compound comprises at least two groups selected from the group consisting of the group represented by the formula (1-1), the group represented by formula (1-2), and the group represented by the formula (1-3).

3. The composition according to claim 1, wherein the compound is represented by formula (2-1) or (2-2): 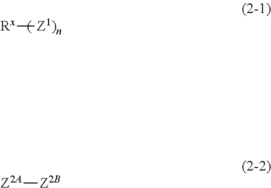
wherein,
in the formula (2-1), Z¹ represents a group having a valency of c, wherein c is an integer of 1 to 3; n is an integer of 1 to 10, wherein in a case in which n is no less than 2, a plurality of Z¹s are identical or different; and R^{X} represents an organic group having 1 to 40 carbon atoms and having a valency of m, wherein m is a sum of valencies for all Z¹s, and
in the formula (2-2), Z^{2A} and Z^{2B} each independently represent a group having a valency of d, wherein d is an integer of 1 to 3.

4. The composition according to claim 1, wherein at least one of p1 and q1 in the formula (1-1), at least one of p2 and q2 in the formula (1-2), and at least one of p3 and q3 in the formula (1-3) are each no less than 1.

5. The composition according to claim 4, wherein R¹, R² or both in each of the formulae (1-1) to (1-3) represents a multiple bond-containing group.

6. A film formed from the composition according to claim 1.

7. A production method of a patterned substrate, the production method comprising:
applying a composition directly or indirectly on at least an upper face of a substrate, to form a resist underlayer film, the composition comprising: a compound comprising at least one group selected from the group consisting of a group represented by formula (1-1), a group represented by formula (1-2), and a group represented by formula (1-3), the compound having a molecular weight of no greater than 3,000; and a solvent;
forming a resist pattern on an upper face side of the resist underlayer film; and
carrying out etching using the resist pattern as a mask, 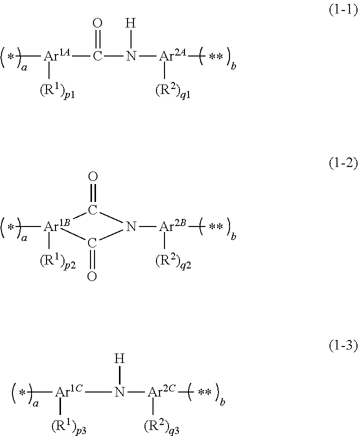
wherein,
in the formulae (1-1) to (1-3), * and ** each denote a site bonding to a part other than the group represented by the formulae (1-1) to (1-3) in the compound; and a and b are each independently an integer of 0 to 3, wherein in a case in which a is 0, b is no less than 1, and in a case in which a is no less than 1, b is 0,
in the formula (1-1), Ar^{1A} represents an aromatic carbocyclic group having 6 to 20 ring atoms and having a valency of (a+p1+1), or an aromatic heterocyclic group having 5 to 20 ring atoms and having a valency of (a+p1+1); R¹ represents a halogen atom, a hydroxy group, a nitro group, or a monovalent organic group having 1 to 20 carbon atoms; p1 is an integer of 0 to 11, wherein in a case in which p1 is no less than 2, a plurality of R¹s are identical or different; Ar^{2A} represents an aromatic carbocyclic group having 6 to 20 ring atoms and having a valency of (b+q1+1), or an aromatic heterocyclic group having 5 to 20 ring atoms and having a valency of (b+q1+1); R² represents a halogen atom, a hydroxy group, a nitro group, or a monovalent organic group having 1 to 20 carbon atoms; q1 is an integer of 0 to 11, wherein in a case in which q1 is no less than 2, a plurality of R²s are identical or different; a sum of p1 and a is no greater than 11; and a sum of q1 and b is no greater than 11,
in the formula (1-2), Ar^{1B} represents an aromatic carbocyclic group having 6 to 20 ring atoms and having a valency of (a+p2+2), or an aromatic heterocyclic group having 5 to 20 ring atoms and having a valency of (a+p2+2); R¹ represents a halogen atom, a hydroxy group, a nitro group, or a monovalent organic group having 1 to 20 carbon atoms; p2 is an integer of 0 to 10, wherein in a case in which p2 is no less than 2, a plurality of R¹s are identical or different; Ar^{2B} represents an aromatic carbocyclic group having 6 to 20 ring atoms and having a valency of (b+q2+1), or an aromatic heterocyclic group having 5 to 20 ring atoms and having a valency of (b+q2+1); q2 is an integer of 0 to 11, wherein in a case in which q2 is 1, R² represents a halogen atom, a hydroxy group, a nitro group, or a monovalent organic group having 1 to 20 carbon atoms, and in a case in which q2 is no less than 2, a plurality of R²s are identical or different, and each R² represents a halogen atom, a hydroxy group, a nitro group, or a monovalent organic group having 1 to 20 carbon atoms, and optionally two or more of the plurality of R²s taken together represent a ring structure having 4 to 20 ring atoms together with the atom chain to which the two or more of the plurality of R²s bond; a sum of p2 and a is no greater than 10; and a sum of q2 and b is no greater than 11, and
in the formula (1-3), Ar^{1C} represents an aromatic carbocyclic group having 6 to 20 ring atoms and having a valency of (a+p3+1), or an aromatic heterocyclic group having 5 to 20 ring atoms and having a valency of (a+p3+1); R¹ represents a halogen atom, a hydroxy group, a nitro group, or a monovalent organic group having 1 to 20 carbon atoms; p3 is an integer of 0 to 11, wherein in a case in which p3 is no less than 2, a plurality of R¹s are identical or different; Ar^{2C} represents an aromatic heterocyclic group having 5 to 20 ring atoms and having a valency of (b+q3+1); R² represents a halogen atom, a hydroxy group, a nitro group, or a monovalent organic group having 1 to 20 carbon atoms; q3 is an integer of 0 to 11, wherein in a case in which q3 is no less than 2, a plurality of R²s are identical or different; a sum of p3 and a is no greater than 11; and a sum of q3 and b is no greater than 11.

8. The production method according to claim 7, wherein the compound comprises at least two groups selected from the group consisting of the group represented by the formula (1-1), the group represented by formula (1-2), and the group represented by the formula (1-3).

9. The production method according to claim 7, wherein the compound is represented by formula (2-1) or (2-2): 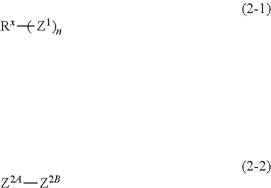
wherein,
in the formula (2-1), Z¹ represents a group having a valency of c, wherein c is an integer of 1 to 3; n is an integer of 1 to 10, wherein in a case in which n is no less than 2, a plurality of Z¹s are identical or different; and R^{X} represents an organic group having 1 to 40 carbon atoms and having a valency of m, wherein m is a sum of valencies for all Z¹s, and
in the formula (2-2), Z^{2A} and Z^{2B} each independently represent a group having a valency of d, wherein d is an integer of 1 to 3.

10. The production method according to claim 7, wherein at least one of p1 and q1 in the formula (1-1), at least one of p2 and q2 in the formula (1-2), and at least one of p3 and q3 in the formula (1-3) are each no less than 1.

11. The production method according to claim 7, wherein R¹, R² or both in each of the formulae (1-1) to (1-3) represents a multiple bond-containing group.
